(19) United States
(12) Patent Application Publication
(10) Pub. No.: US 2002/0123147 A1
(43) Pub. Date: Sep. 5, 2002

(54) Gene transfer with adenoviruses having modified fiber proteins

(75) Inventors: Alan McClelland, Gaithersburg, MD (US); Susan C. Stevenson, Frederick, MD (US); Mario Gorziglia, Gaithersburg, MD (US); Elio F. Vanin, Memphis, TN (US)

Correspondence Address: ROTHWELL, FIGG, ERNST & MANBECK, P.C., 1425 K STREET, N.W., SUITE 800, WASHINGTON, DC, 20005, US

(73) Assignee: GENETIC THERAPY, INC., Gaithersburg, MD (US)

(21) Appl. No.: 09/993,502

(22) Filed: Nov. 27, 2001

Related U.S. Application Data
(63) Continuation of application No. 08/852,924, filed on May 8, 1997, now abandoned.

(51) Int. Cl. C12N 15/861; C12N 7/01

(52) U.S. Cl. 435/456; 435/235.1

(57) ABSTRACT

A method of transferring at least one DNA sequence into cells by transducing the cells, in vivo or ex vivo, with a modified adenovirus. The adenovirus, prior to modification, is of a first serotype. In the modified adenovirus, at least a portion of the fiber, and in particular the head portion, is removed from the adenovirus of the first serotype and replaced with a portion, in particular the head portion, of the fiber of an adenovirus of a second serotype. Such method is useful in transducing cells which may be refractory to the adenovirus of the first serotype, yet include a receptor which binds to the head portion of the fiber of the adenovirus of the second serotype.

This application is a continuation of U.S. Ser. No. 08/852,924, filed May 8, 1997.

This invention relates to adenoviruses as used as gene delivery vehicles, whereby genes are transferred into cells. More particularly, the invention relates to the transfer of genes into cells by employing a modified adenovirus. The adenovirus, prior to modification, is of a first serotype, and the adenovirus is modified such that at least a portion, preferably the head portion, of the fiber of the adenovirus of the first serotype is removed and replaced with at least a portion, preferably the head portion, of the fiber of an adenovirus of a second serotype.

This invention also relates to gene delivery or gene transfer vehicles other than adenoviruses, which have been modified to include at least a portion, preferably the head portion, of the fiber of an adenovirus of a desired serotype, whereby the gene delivery or gene transfer vehicle will bind to a receptor for the portion of the fiber, preferably the head portion, of the adenovirus of the desired serotype. Such gene delivery or gene transfer vehicles may be viruses, such as, for example, retroviruses, adeno-associated virus, and Herpes viruses, which have a viral surface protein which has been modified to include at least a portion of the fiber, preferably the head portion, of the fiber of an adenovirus of a desired serotype. Alternatively, the gene delivery or gene transfer vehicle may be a non-viral gene delivery or gene transfer vehicle, such as a plasmid, to which is bound at least a portion, preferably the head portion, of the fiber of an adenovirus of a desired serotype. In another example, the gene delivery or gene transfer vehicle may be a proteoliposome which encapsulates an expression vehicle, wherein the proteoliposome includes a portion, preferably the head portion, of the fiber of an adenovirus of a desired serotype.

This invention further relates to adenoviruses of the Adenovirus 3 serotype which include at least one heterologous DNA sequence, and to the transfer of polynucleotides into cells which include a receptor which binds to the head portion of the fiber of Adenovirus 3, by contacting such cells with a gene transfer vehicle which includes the head portion of the fiber of Adenovirus 3. The term “gene transfer vehicle,” as used herein, means any construct which is capable of delivering a polynucleotide (DNA or RNA) sequence to a cell. Such gene transfer vehicles include, but are not limited to, viruses, such as adenoviruses, retroviruses, adeno-associated virus, Herpes viruses, plasmids, proteoliposomes which encapsulate a polynucleotide sequence to be transferred into a cell, and “synthetic viruses” and “synthetic vectors” which include a polynucleotide which is enclosed within a fusogenic polymer layer, or within an inner fusogenic polymer layer and an outer hydrophilic polymer layer.

The term “polynucleotide” as used herein means a polymeric form of nucleotide of any length, and includes ribonucleotides and deoxyribonucleotides. Such term also includes single- and double-stranded RNA. The term also includes modified polynucleotides such as methylated or capped polynucleotides.

### BACKGROUND OF THE INVENTION

Adenovirus genomes are linear, double-stranded DNA molecules about 36 kilobase pairs long. Each extremity of the viral genome has a short sequence known as the inverted terminal repeat (or ITR), which is necessary for viral replication. The well-characterized molecular genetics of adenovirus render it an advantageous vector for gene transfer. The knowledge of the genetic organization of adenoviruses allows substitution of large fragments of viral DNA with foreign sequences. In addition, recombinant adenoviruses are stable structurally, and no rearranged viruses are observed after extensive amplification.

Adenoviruses may be employed as delivery vehicles for introducing desired genes into eukaryotic cells. The adenovirus delivers such genes to eukaryotic cells by binding cellular receptors. The adenovirus fiber protein is responsible for such attachment. (Philipson, et al., J. Virol., Vol. 2, pgs. 1064-1075 (1968)). The fiber protein includes a tail portion, a shaft portion, and a globular head portion which contains the putative receptor binding region. The fiber spike is a homotrimer, and there are 12 spikes per virion.

In susceptible cells, the adenoviral cellular entry pathway is an efficient process which involves two separate cell surface events (Wickham, et al., Cell, Vol. 73, pgs, 309-319 (1993)). First, a high affinity interaction between the adenoviral capsid fiber protein and an unidentified cell surface receptor mediates the attachment of the adenoviral particle to the cell surface. A subsequent association of the penton with the cell surface integrins, αvβ3 and αvβ5 which act as co-receptors, potentiate virus internalization (Wickham, 1993). Competition binding experiments using intact adenoviral particles and expressed fiber proteins have provided evidence for the existence of at least two distinct denoviral fiber receptors which interact with the subgenus B (Adenovirus 3) and subgenus C (Adenovirus 5) adenoviruses (Defer, et al., J. Virol., Vol. 64, 3661-3673 (1990); Mathias, et al., J. Virol., Vol. 68, pgs. 6811-6814 (1994); Stevenson, et al., J. Virol., Vol. 69, pgs. 2650-2857 (1995)). Although Adenovirus 5 and Adenovirus 3 utilize different fiber binding receptors, αv integrins enhance entry of both serotypes into cells (Mathias, 1994). This suggests that the binding and entry steps are unlinked events and that fiber attachment to various cell surface molecules may permit productive entry. It is likely that additional receptors exist for other adenoviral serotypes although this remains to be demonstrated.

Adenoviral vectors derived from the human Subgenus C, Adenovirus 5 serotype are efficient gene delivery vehicles which readily transduce many nondividing cells. Adenoviruses infect a broad range of cells and tissues including lung, liver, endothelium, and muscle (Trapnell, et al. Curr. Opinion Biotech., Vol. 5, pgs. 617-625 (1994). High titer stocks of purified adenoviral vectors can be prepared which makes the vector suitable for in vivo administration. Various routes of in vivo administration have been investigated including intravenous delivery for liver transduction and intratracheal instillation for gene transfer to the lung. As the adenoviral vector system is more widely applied, it is becoming apparent that some cell types may be refractory to recombinant adenoviral infection. Both the fiber binding receptor and αvβ3 or αvβ5 integrins are important for high efficiency infection of target cells. Efficient transduction requires fiber mediated attachment as demonstrated by the effectiveness of recombinant soluble fiber in blocking gene transfer (Goldman, et al., J. Virol., Vol. 69, pgs. 5951-5958 (1995)). Transduction of cells which lack fiber receptors occurs with much lower efficiency and requires high multiplicities of input vector (Freimuth, et al., J. Virol., Vol. 70, pgs. 4081-4085 (1996); Haung, et al., J. Virol., Vol. 70, pgs. 4502-4508 (1996)). Fiber independent transduction likely occurs Through direct binding of the penton base arginine-glycine-aspartic acid, or RGD, sequences to cell surface integrins. Blockade of the RGD:integrin pathway reduces gene transfer efficiencies by several fold (Freimuth, 1996; Haung, 1996), but the effect is less complete than blockade of the fiber receptor interaction, suggesting that the latter is more critical.

Low level gene transfer may result from a deficiency in one of the components of the entry process in the target cell. For example, inefficient gene transfer to human pulmonary epithelia has been attributed to a deficiency in avb5 integrins (Goldman, 1995). Other cell types such as vascular endothelial and smooth muscle cells have been identified as being deficient in fiber dependent transduction due to a low level of the Adenovirus 5 receptor (Wickham, et al., J. Virol., Vol. 70, pgs. 6831-6838 (1996)). Several approaches have been undertaken to target adenoviral vectors to improve or enable efficient transduction of target cells. These strategies include alteration of the penton base to target selectively specific cell surface integrins (Wickham, et al., Gene Ther., Vol. 2, pgs. 750-756 (1995); Wickham, et al., J. Virol., Vol. 70, pgs. 6831-6838 (1996)) and modification of the fiber protein with an appropriate ligand to redirect binding (Michael, et al., Gene Ther., Vol. 2, pgs. 660-668 (1995); Stevenson, 1995).

### SUMMARY OF THE INVENTION

The present invention is directed to the transduction of cells with adenoviruses wherein at least a portion of the fiber of the adenovirus, and in particular the head portion, is removed and replaced with a fiber portion, and in particular, a head portion of the fiber, having novel receptor specificities. Binding of recombinant Adenovirus 5 and Adenovirus 3 fiber proteins to cellular receptors has been examined previously, and it was demonstrated that the receptor specificity of the fiber protein can be altered by exchanging the head domains between these two fiber proteins (Stevenson, 1995). Thus, the present invention is directed to the transduction of cells with a modified adenovirus having a chimeric fiber, wherein the adenovirus, prior to modification, is of a first serotype, and the adenovirus is modified such that at least a portion of the fiber, and in particular the head portion, of the adenovirus is removed and replaced with at least a portion of the fiber of an adenovirus of the second serotype. Applicants have found that such adenoviruses bind to cells having a receptor for the adenovirus of the second serotype. Applicants also have found that such adenoviruses may bind to cells which are refractory to adenoviruses of the first serotype, yet are bound by the modified adenoviruses through the binding of the head region of the fiber of the modified adenovirus to a receptor for the adenovirus of the second serotype.

The present invention also is directed to gene delivery or gene transfer vehicles, other than adenoviruses, which include at least a portion, preferably the head portion, of the fiber of an adenovirus of a desired serotype. Such gene transfer vehicles are useful for delivering polynucleotides to cells which have a receptor that binds to the fiber of the adenovirus of a desired serotype. The gene transfer vehicles which may be employed include, but are not limited to, retroviruses, adeno-associated virus, Herpes viruses, plasmids which are linked chemically to the at least a portion of the fiber of the adenovirus of a desired serotype, and proteoliposomes encapsulating the polynucleotide which is to be transferred into cells.

In yet another embodiment, the present invention is directed to an adenovirus of the Adenovirus 3 serotype which includes at least one heterologous DNA sequence.

In a further embodiment, the present invention also is directed to the transfer of polynucleotides into cells which include a receptor for Adenovirus 3 by contacting such cells with a gene transfer vehicle including at least a portion, and preferably the head portion, of the giber of Adenovirus 3.

### BRIEF DESCRIPTION OF THE DRAWINGS

The invention now will be described with respect to the drawings, wherein:

FIG. 1 shows genomic analysis of the wild type fiber, Av1LacZ4 and chimeric fiber, Av9LacZ4 adenoviral vectors. FIG. 1A shows ScaI (S), DraI (D), EcoRI (E) and BamHI (B) restriction endonuclease sites on a schematic diagram for each vector. The predicted DraI and ScaI restriction fragments and the expected sizes for Av1LacZ4 and Av9LacZ4 are highlighted. DNA was isolated from each vector, digested with the indicated restriction endonucleases, and Southern blot analysis carried out using standard procedures. FIG. 1B shows digested DNA samples (0.4 μg) that were applied to a 0.8% agarose gel and stained with ethidium bromide to visualize the individual DNA fragments. The combined λDNA/HindIII and φX174 RF DNA/HaeIII DNA size markers (M) are indicated. The Av1LacZ4 wildtype vector was digested with: lane 1, ScaI; lane 2, DraI; and lane 3, EcoRI and BamHI. The Av9LacZ4 chimeric fiber vector was digested with: lane 4, ScaI; lane 5, DraI and lane 6, EcoRI and BamHI. FIG. 1C shows digested DNA fragments as shown in FIG. 1B that were transferred to a Zetaprobe membrane and hybridized with the [³²P]-labeled 500 bp Adenovirus 3 fiber head domain probe at approximately 1×10⁶ cpm/ml and exposed to film for 12 hours. The expected fragments derived from Av9LacZ4 which hybridized with the Adenovirus 3 fiber head probe are indicated.

FIG. 2 shows Western immunoblot analysis of adenoviral capsid proteins. An equivalent number of adenoviral particles for the Av1LacZ4 (lanes 1 and 4), Av9LacZ4 (lanes 2 and 5) vectors or a control virus containing the full length Adenovirus 3 fiber protein (lanes 3 and 6) were subjected to 4/15% SDS PAGE and Western blot analysis under denaturing conditions. (A) 2×10¹⁰ adenoviral particles were applied per lane and the membrane was developed with the anti-fiber monoclonal antibody, 4D2-5 and an anti-mouse IgG-HRPO conjugated secondary antibody by chemiluminescence. (B) 6×10¹⁰ particles were applied per lane and the membrane was developed using a rabbit anti-Adenovirus 3 fiber specific polyclonal antibody and donkey anti-rabbit IgG-HRPO secondary antibody by chemiluminescence. The positions of molecular weight markers are indicated.

FIGS. 3A and 3B are graphs of the results of competition viral transduction assays. HeLa cell monolayers were incubated with increasing concentrations of purified Adenovirus 5 fiber trimer protein (5F, FIG. 3A) or with an insect cell lysate containing the Adenovirus 3 fiber protein (3F/CL, FIG. 3B) prior to transduction with 100 total particles per cell of either the Av1LacZ4 (open circles) or Av9LacZ4 (closed circles) adenoviral vectors. After 24 hours, the cells were analyzed for β-galactosidase expression as described in Example 1. The percentage of adenoviral transduction at each concentration of competitor is plotted. Each point is the average ± standard deviation of three independent determinations for a representative experiment.

FIG. 4 shows differential adenoviral-mediated transduction properties of human cell lines. HeLa (FIGS. 4A and 4B), MRC-5 (FIGS. 4C and 4D), and FaDu (FIGS. 4E and 4F) cells were transduced with the Av1LacZ4 (FIGS. 4A, 4C, and 4E) or Av9LacZ4 (FIGS. 4B, 4D, and 4F) vectors at 1000 total particles per cell. After 24 hours the cells were analyzed for β-galactosidase expression as described in Example 1. Representative photomicrographs are shown.

FIGS. 5A, 5B, and 5C are graphs showing Adenoviral-mediated transduction properties of HeLa, MRC-5, and FaDu human cell lines. The indicated cells were transduced with 0,10,100, and 1000 total particles per cell of the Av1LacZ4 (open circles) or Av9LacZ4 (closed circles) vectors for one hour at 37° C. in a total volume of 0.2 ml of culture medium. After 24 hours, the cells were fixed and stained with X-gal as described in Example 1. The percent transduced cells per high power field was determined for each vector dose. The data represent the average percent transduction ± standard deviation for three independent experiments and each vector dose was carried out in triplicate. The percentage transduction of HeLa (FIG. 5A), MRC-5 (FIG. 5B) and FaDu (FIG. 5C) cells at each vector dose is displayed.

FIGS. 6A and 6B are graphs showing differential adenoviral-mediated transduction properties of human cell lines. The percent transduction efficiency for each cell line infected with the Av1LacZ4 (open bars) or Av9LacZ4 (closed bars) vectors is displayed for the vector dose of 100 (FIG. 6A) and 1000 (FIG. 6B) particles per cell. The data represent the mean ± standard deviation from three independent experiments. The cell lines are as follows: HeLa: human cervical carcinoma cells; HDF: human diploid fibroblasts; THP-1: human monocytes; MRC-5: human embryonic lung diploid fibroblasts; FaDu: human squamous carcinoma cells; HUVEC: human umbilical vein endothelial cells, and HCAEC: human coronary artery endothelial cells.

### DETAILED DESCRIPTION OF THE INVENTION

In accordance with an aspect of the present invention, there is provided a method of transferring at least one DNA sequence into cells. The method comprises transducing the cells with a modified adenovirus including the at least one DNA sequence. The adenovirus, prior to modification, is of a first serotype. In the modified adenovirus, at least a portion of the fiber of the adenovirus is removed and replaced with at least a portion of the fiber of an adenovirus of a second serotype. The cells include a receptor which binds to the at least a portion of the fiber of the adenovirus of the second serotype. Transfer of the at least one DNA sequence into said cells is effected through binding of the modified adenovirus to the cells.

As stated hereinabove, the adenovirus fiber protein includes a head portion, a shaft portion, and a tail portion. In one embodiment, at least a portion of the head portion of the fiber of the adenovirus of the first serotype is removed and replaced with at least a portion of the head portion of the adenovirus of the second serotype. In a preferred embodiment, all of the head portion of the fiber of the adenovirus of the first serotype is removed and replaced with the head portion of the fiber of the adenovirus of the second serotype.

In one embodiment, the first and second serotypes of the adenoviruses are from different subgenera. In general, the human adenoviruses are divided into Subgenera A through F. Such subgenera are described further in Bailey, et al., Virology, Vol. 205, pgs. 438-452 (1994), the contents of which are herein incorporated by reference. Subgenus A includes Adenovirus 12, Adenovirus 18 and Adenovirus 31. Subgenus B includes Adenovirus 3, Adenovirus 7, Adenovirus 14, and Adenovirus 35. Subgenus C includes Adenovirus 1, Adenovirus 2, Adenovirus 5, and Adenovirus 6. Subgenus D includes Adenovirus 9, Adenovirus 10, Adenovirus 15, and Adenovirus 19. Subgenus E includes Adenovirus 4. Subgenus F includes Adenovirus 40 and Adenovirus 41. In one embodiment, the adenovirus of the first serotype is an Adenovirus of a serotype within Subgenus C, and the adenovirus of the second serotype is an adenovirus of a serotype within a subgenus selected from the group consisting of Subgenera A, B, D, E, and F. In another embodiment, the adenovirus of the second serotype is an adenovirus of a serotype within Subgenus B. In yet another embodiment, the adenovirus of the first serotype is Adenovirus 5, and the adenovirus of the second serotype is Adenovirus 3. Thus, in such embodiment, amino acid residues 404 to 581 of the fiber (i.e., the fiber head region) of Adenovirus 5 are removed and replaced with amino acid residues 136 to 319 of the fiber (i.e., the fiber head region) of Adenovirus 3. The DNA encoding the fiber protein of Adenovirus 5 is registered as Genbank Accession No. M18369 (incorporated herein by reference), and the DNA encoding the fiber protein of Adenovirus 3 is registered as Genbank Accession No. M12411.

Cells which may be transduced with the modified adenovirus include those cells which have a receptor that binds to the portion of the fiber protein, and in particular the head portion of the fiber protein, of the adenovirus of the second serotype. When the modified adenovirus is an adenovirus of the Adenovirus 5 serotype having a fiber head portion of Adenovirus 3, the cells which may be transduced by such modified adenovirus include, but are not limited to, lung cells, including, but not limited to, lung epithelial cells and lung cancer cells; blood cells such as hematopoietic cells, including, but not limited to, monocytes and macrophages; lymphoma cells; leukemia cells, including acute myeloid leukemia cells and acute lymphocytic leukemia cells; smooth muscle cells, including, but not limited to, smooth muscle cells of blood vessels and of the digestive system; and tumor cells, including, but not limited to, head and neck cancer cells and neuroblastoma cells.

Such adenoviruses may be constructed from an adenoviral vector of a first serotype wherein DNA encoding at least a portion of the fiber is removed and replaced with DNA encoding at least a portion of the fiber of the adenovirus of a second serotype.

The adenovirus, in general, also includes at least one DNA sequence to be transferred into cells. The at least one DNA sequence may be a heterologous DNA sequence, and in particular, may be a heterologous DNA sequence encoding a therapeutic agent. The term “therapeutic” is used in a generic sense and includes treating agents, prophylactic agents, and replacement agents.

DNA sequences encoding therapeutic agents include, but are not limited to, DNA sequences encoding tumor necrosis factor (TNF) genes, such as TNF-α; genes encoding interferons such as Interferon-α, Interferon-β, and Interferon-γ; genes encoding interleukins such as IL-1, IL-1β, and Interleukins 2 through 14; genes encoding G-CSF, GM-CSF, TGF-α, TGF-β, and fibroblast growth factor; genes encoding ornithine transcarbamylase, or OTC; genes encoding adenosine deaminase, or ADA; genes which encode cellular growth factors, such as lymphokines, which are growth factors for lymphocytes; genes encoding epidermal growth factor (EGF), vascular endothelial growth factor (VEGF), and keratinocyte growth factor (KGF); genes encoding soluble CD4; Factor VIII; Factor IX; cytochrome b; glucocerebrosidase; T-cell receptors; the LDL receptor, ApoE, ApoC, ApoAI and other genes involved in cholesterol transport and metabolism; the alpha-1 antitrypsin (α1AT) gene; genes encoding co-stimulatory antigens, such as B7.1; genes encoding chemotactic agents, such as lymphotactin, the cystic fibrosis transmembrane conductance regulator (CFTR) genes; the insulin gene; the hypoxanthine phosphoribosyl transferase gene; negative selective markers or “suicide” genes, such as viral thymidine kinase genes, such as the Herpes Simplex Virus thymidine kinase gene, the cytomegalovirus virus thymidine kinase gene, and the varicella-zoster virus thymidine kinase gene; Fc receptors for antigen-binding domains of antibodies, antisense sequences which inhibit viral replication, such as antisense sequences which inhibit replication of hepatitis B or hepatitis non-A non-B virus; antisense c-myb oligonucleotides; and antioxidants such as, but not limited to, manganese superoxide dismutase (Mn-SOD), catalase, copper-zinc-superoxide dismutase (CuMn-SOD), extracellular superoxide dismutase (EC-SOD), and glutathione reductase; tissue plasminogen activator (tPA); urinary plasminogen activator (urokinase); hirudin; the phenylalanine hydroxylase gene; nitric oxide synthetase; vasoactive peptides; angiogenic peptides; the dopamine gene; the dystrophin gene; the β-globin gene; the α-globin gene; the HbA gene; protooncogenes such as the ras, src, and bcl genes; tumor-suppressor genes such as p53 and Rb; genes encoding anti-angiogenic factors, such as, for example, endothelial monocyte activating polypeptide-2 (EMAP-2); the heregulin-α protein gene, for treating breast, ovarian, gastric and endometrial cancers; cell cycle control agent genes, such as, for example, the p21 gene; antisense polynucleotides to the cyclin G1 and cyclin D1 genes; the endothelial nitric oxide synthetase (ENOS) gene; moroclonal antibodies specific to epitopes contained within the β-chain of a T-cell antigen receptor; the multidrug resistance (MDR) gene; the dihydrofolate reductase (DHFR) gene; DNA sequences encoding ribozymes; antisense polynucleotides; genes encoding secretory peptides which act as competitive inhibitors of angiotensin converting enzyme, of vascular smooth muscle calcium channels, or of adrenergic receptors, and DNA sequences encoding enzymes which break down amyloid plaques within the central nervous system. It is to be understood, however, that the scope of the present invention is not to be limited to any particular therapeutic agent.

The DNA sequence which encodes the therapeutic agent may be genomic DNA or may be a cDNA sequence. The DNA sequence also may be the native DNA sequence or an allelic variant thereof. The term “allelic variant” as used herein means that the allelic variant is an alternative form of the native DNA sequence which may have a substitution, deletion, or addition of one or more nucleotides, which does not alter substantially the function of the encoded protein or polypeptide or fragment or derivative thereof. In one embodiment, the DNA sequence may further include a leader sequence or portion thereof, a secretory signal or portion thereof and/or may further include a trailer sequence or portion thereof.

The DNA sequence encoding at least one therapeutic agent is under the control of a suitable promoter. Suitable promoters which may be employed include, but are not limited to, adenoviral promoters, such as the adenoviral major late promoter; or heterologous promoters, such as the cytomegalovirus (CMV) promoter; the Rous Sarcoma Virus (RSV) promoter; inducible promoters, such as the MMT promoter, the metallothionein promoter; heat shock promoters; the albumin promoter; and the ApoAI promoter. It is to be understood, however, that the scope of the present invention is not to be limited to specific foreign genes or promoters.

The adenoviral vector which is employed may, in one embodiment, be an adenoviral vector which includes essentially the complete adenoviral genome (Shenk et al., Curr. Top. Microbiol. Immunol., 111(3): 1-39 (1984). Alternatively, the adenoviral vector may be a modified adenoviral vector in which at least a portion of the adenoviral genome has been deleted.

In a preferred embodiment, the adenoviral vector comprises an adenoviral 5′ ITR; an adenoviral 3′ ITR; an adenoviral encapsidation signal; a DNA sequence encoding a therapeutic agent; and a promoter controlling the DNA sequence encoding a therapeutic agent. The vector is free of at least the majority of adenoviral E1 and E3 DNA sequences, but is not free of all of the E2 and E4 DNA sequences, and DNA sequences encoding adenoviral proteins promoted by the adenoviral major late promoter.

In one embodiment, the vector also is free of at least a portion of at least one DNA sequence selected from the group consisting of the E2 and E4 DNA sequences.

In another embodiment, the vector is free of at least the majority of the adenoviral E1 and E3 DNA sequences, and is free of at least a portion of the other of the E2 and E4 DNA sequences.

In still another embodiment, the gene in the E2a region that encodes the 72 kilodalton binding protein is mutated to produce a temperature sensitive protein that is active at 32° C., the temperature at which the viral particles are produced. This temperature sensitive mutant is described in Ensinger et al., J. Virology, 10:328-339 (1972), Van der Vliet et al., J. Virology, 15:348-354 (1975), and Friefeld et al., Virology, 124:380-389 (1983).

Such a vector, in a preferred embodiment, is constructed first by constructing, according to standard techniques, a shuttle plasmid which contains, beginning at the 5′ end, the “critical left end elements,” which include an adenoviral 5′ ITR, an adenoviral encapsidation signal, and an E1a enhancer sequence; a promoter (which may be an adenoviral promoter or a foreign promoter); a multiple cloning site (which may be as herein described); a poly A signal; and a DNA segment which corresponds to a segment of the adenoviral genome. The vector also may contain a tripartite leader sequence. The DNA segment corresponding to the adenoviral genome serves as a substrate for homologous recombination with a modified or mutated adenovirus, and such sequence may encompass, for example, a segment of the adenovirus 5 genome no longer than from base 3329 to base 6246 of the genome. The plasmid may also include a selectable marker and an origin of replication. The origin of replication may be a bacterial origin of replication. Representative examples of such shuttle plasmids include pAvS6, which is described in published PCT Application Nos. WO94/23582, published Oct. 27, 1994, and WO95/09654, published Apr. 13, 1995 and in U.S. Pat. No. 5,543,328, issued Aug. 6, 1996. The DNA sequence encoding a therapeutic agent then may be inserted into the multiple cloning site to produce a plasmid vector.

This construct is then used to produce an adenoviral vector. Homologous recombination is effected with a modified or mutated adenovirus in which at least the majority of the E1 and E3 adenoviral DNA sequences have been deleted. Such homologous recombination may be effected through co-transfection of the plasmid vector and the modified adenovirus into a helper cell line, such as 293 cells, by CaPO₄ precipitation. Upon such homologous recombination, a recombinant adenoviral vector is formed that includes DNA sequences derived from the shuttle plasmid between the Not I site and the homologous recombination fragment, and DNA derived from the E1 and E3 deleted adenovirus between the a homologous recombination fragment and the 3′ ITR.

In one embodiment, the homologous recombination fragment overlaps with nucleotides 3329 to 6246 of the Adenovirus 5 (ATCC VR-5) genome.

Through such homologous recombination, a vector is formed which includes an adenoviral 5′ ITR, an adenoviral encapsidation signal; an E1a enhancer sequence; a promoter; at least one DNA sequence encoding a therapeutic agent; a poly A signal; adenoviral DNA free of at least the majority of the E1 and E3 adenoviral DNA sequences; and an adenoviral 3′ ITR. The vector also may include a tripartite leader sequence. The vector may then be transfected into a helper cell line, such as the 293 helper cell line (ATCC No. CRL1573), which will include the E1a and E1b DNA sequences, which are necessary for viral replication, and to generate adenoviral particles. Transfection may take place by electroporation, calcium phosphate precipitation, microinjection, or through proteoliposomes.

In another embodiment, the adenoviral vector is free of all or a portion of each of the adenoviral E1 and E4 DNA sequences, or is free of all or a portion of each of the adenoviral E1 and E2 DNA Sequences, or is free of all or a portion of each of the E1, E2, and E4 DNA sequences.

Such vectors may be assembled by direct in vitro ligation from combinations of plasmids containing portions of modified or unmodified virus genome or plasmids and fragments derived directly from a linear adenoviral genome, such as the Adenovirus 5 genome (ATCC No. VR-5) or Adenovirus 5 derived viruses containing mutations or deletions.

In another alternative, the vectors can be assembled by homologous recombination, within a eukaryotic cell, between a plasmid clone containing a portion of the adenoviral genome (such as the Adenovirus 5 genome or the adenovirus 5 E3-mutant Ad d1327 (Thimmapaya, et al., Cell, Vol. 31, pg. 543 (1983)) with the desired modifications, and a second plasmid (such as, for example pAvS6), containing the left adenoviral ITR, an E1 region deletion, and the desired trans gene. Alternatively, homologous recombination may be carried out between a plasmid clone and a fragment derived directly from a linear adenovirus (such as Adenovirus 5, or Ad d1327 or an Adenovirus 5 derived virus containing mutations or deletions) genome.

The vector then is transfected into a cell line capable of complementing the function of any essential genes deleted from the viral vector, in order to generate infectious viral particles. The cell line in general is a cell line which is infectable and able to support adenovirus or adenoviral vector growth, provide for continued virus production in the presence of glucocorticoid hormones, and is responsive to glucocorticoid hormones (i.e., the cell line is capable of expressing a glucocorticoid hormone receptor). Cell lines which may be transfected with the essential adenoviral genes, and thus may be employed for generating the infectious adenoviral particles include, but are not limited to, the A549, KB, and Hep-2 cell lines.

Because the expression of some viral genes may be toxic to cells, the E1 region, as welt as the E2a, E2b, and/or E4 regions, may be under the control of an inducible promoter. Such inducible promoters may include, but are not limited to, the mouse mammary tumor virus (MMTV) promoter (Archer, et al., Science, Vol. 255, pgs. 1573-1576 (Mar. 20, 1992)); the synthetic minimal glucocorticoid response element promoter GRE5 (Mader, et al., Proc. Nat. Acad. Sci., Vol. 90, pgs. 5603-5607 (June 1993)); or the tetracycline-responsive promoters (Gossen, et al., Proc. Nat. Acad. Sci., Vol. 89, pgs. 5547-5551 (June 1992)). In another alternative, the E1 region is under the control of an inducible promoter, and the E2a, E2b and/or E4 regions are under the control of their native promoters. In such alternative, the native promoters are transactivated by expression of the E1 region.

In one embodiment, the cell line includes the entire adenoviral E4 region with its native promoter region, and the E1a region or the entire E1 region (including the E1a and E1b regions) under the control of a regulatable or inducible promoter, such as, for example, the mouse mammary tumor virus (or MMTV) promoter, which is a hormone inducible promoter, or other such promoters containing glucocorticoid responsive elements (GRE's) for transcriptional control. In another embodiment, the E4 DNA sequence also is expressed from a regulatable promoter, such as the MMTV promoter. The E1 and E4 DNA sequences may be included in one expression vehicle, or may be included in separate expression vehicles. Preferably, the expression vehicles are plasmid vectors which integrate with the genome of the cell line.

Such vectors, wherein the vector is free of all or a portion of each of the adenoviral E1 and E4 DNA sequences, or is free of all or a portion of each of the adenoviral E1 and E2 DNA sequences, or is free of all or a portion of the E1, E2, and E4 DNA sequences, and the complementing cell lines, also are described in PCT Application No. WO96/18418, published Jun. 20, 1996, the contents of which are incorporated herein by reference.

Upon formation of the adenoviral vectors hereinabove described, the genome of such a vector is modified such that DNA encoding at least a portion of the fiber protein is removed and replaced with DNA encoding at least a portion of the fiber protein an adenovirus having a serotype different from that of the adenovirus being modified. Such modification may be accomplished through genetic engineering techniques known to those skilled in the art.

Upon modification of the genome of the adenoviral vector, the vector is transfected into an appropriate cell line for the generation of infectious adenoviral particles wherein at least a portion of the fiber protein, in particular the head portion has been changed to include a portion, and in particular the head portion, of the fiber protein of an adenovirus having a serotype different from that of the adenovirus being modified.

Alternatively, the DNA sequence encoding the modified fiber may be placed into an adenoviral shuttle plasmid such as those hereinabove described. The shuttle plasmid also may include at least one DNA sequence encoding a therapeutic agent. The shuttle plasmid is transfected into an appropriate cell line for the generation of infectious viral particles, with an adenoviral genome wherein the DNA encoding the fiber protein is deleted.

In another alternative, a first shuttle plasmid includes at least one DNA sequence encoding the therapeutic agent, and a second shuttle plasmid includes the DNA sequence encoding the modified fiber. The first shuttle plasmid is transfected into an appropriate cell line for the generation of infectious viral particles including at least one DNA sequence encoding a therapeutic agent. The second shuttle plasmid, which includes the DNA sequence encoding the modified fiber, then is transfected with the adenovirus including the at least one DNA sequence encoding a therapeutic agent into an appropriate cell line to generate infectious viral particles including the modified fiber and DNA sequence encoding at least one therapeutic agent through homologous recombination.

In yet another alternative, the modified adenovirus is constructed by effecting homologous recombination between an adenoviral vector of the first serotype which includes at least one DNA sequence encoding a therapeutic agent, with a shuttle plasmid including a DNA sequence encoding the modified fiber.

The modified adenovirus may be employed to transduce cells in vivo, ex vivo, or in vitro. When administered in vivo, the adenoviruses of the present invention may be administered in an amount effective to provide a therapeutic effect in a host. In one embodiment, the modified adenovirus may be administered in an amount of from 1 plaque forming unit to about 10¹⁴ plaque forming units, preferably from about 10⁶ plaque forming units to about 10¹³ plaque forming units. The host may be a mammalian host, including human or non-human primate hosts.

The modified adenovirus may be administered in combination with a pharmaceutically acceptable carrier suitable for administration to a patient, such as, for example, a liquid carrier such as a saline solution, protamine sulfate (Elkins-Sinn, Inc., Cherry Hill, N.J.), or Polybrene (Sigma Chemical).

Cells which may be transduced with the modified adenovirus are those which include a receptor for the adenovirus of the second serotype, whereby the portion of the fiber of the adenovirus of the second serotype, in particular the head portion, which is included in the modified adenovirus, is bound by the receptor for the adenovirus of the second serotype.

When, as in one embodiment, the idenovirus of the first serotype is Adenovirus 5, and such adenovirus has been modified such that at least a portion of the fiber, in particular the head portion of Adenovirus 5, has been removed and replaced with at least a portion, in particular the head portion of Adenovirus 3, cells which may be transduced include lung cells, including normal lung cells such as lung epithelial cells, lung fibroblasts, and lung cancer cells; blood cells, such as hematopoietic cells, including monocytes and macrophages; lymphoma cells; leukemia cells, including acute myeloid leukemia cells and acute lymphocytic leukemia cells; smooth muscle cells, including smooth muscle cells of blood vessels and of the digestive system; and tumor cells, including head and neck cancer cells, lung cancer cells, and neuroblastoma cells.

Thus, a modified adenovirus of the Adenovirus 5 serotype which includes a head portion of the fiber of Adenovirus 3 may be used to treat a disease or disorder of the lung (such as, for example, cystic fibrosis, lung surfactant protein deficiency states, or emphysema). The modified adenovirus may be administered, for example, by aerosolized inhalation or bronchoscopic installation, or via intranasal or intratracheal instillation.

For example, the modified adenoviruses may be used to infect lung cells, and such modified adenoviruses may include the CFTR gene, which is useful in the treatment of cystic fibrosis. In another embodiment, the modified adenovirus may include a gene(s) encoding a lung surfactant protein, such as surfactant protein A (SP-A), surfactant protein B (SP-B), or surfactant protein C (SP-C), whereby the modified adenoviral vector is employed to treat lung surfactant protein deficiency states. In yet another embodiment, the modified adenovirus may include a gene encoding α-1-antitrypsin, whereby the modified adenovirus may be employed in the treatment of emphysema caused by α-1-antitrypsin deficiency.

In another embodiment, the modified adenoviruses may be used to infect hematopoietic stem cells of a cancer patient undergoing chemotherapy in order to protect such cells from adverse effects of chemotherapeutic agents. Such cells may be transduced with the modified adenovirus in vivo, or the cells may be obtained from a blood sample or bone marrow sample that is removed from the patient, transduced with the modified adenovirus ex vivo, and returned to the patient. For example, hematopoietic stem cells may be transduced in vivo or ex vivo with a modified adenovirus of the present invention which includes a multidrug resistance (MDR) gene or a dihydrofolate reductase (DHFR) gene. Such transduced hematopoietic stem cells become resistant to chemotherapeutic agents, and therefore such transduced hematopoietic stem cells can survive in cancer patients that are being treated with chemotherapeutic agents.

In yet another embodiment, the modified adenoviruses may be employed in the treatment of tumors, such as head and neck cancer, neuroblastoma, lung cancer, and lymphomas. For example, the modified adenovirus may include a negative selective marker, or “suicide” gene, such as the Herpes Simplex Virus thymidine kinase (TK) gene. The modified adenovirus may be employed in the treatment of the head and neck cancer or lung cancer, or neuroblastoma, or lymphoma, by administering the modified adenovirus to a patient, such as, for example, by direct injection of the modified adenovirus into the tumor or into the lymphoma, whereby the modified adenovirus transduces the tumor cells or lymphoma cells. Alternatively, when the modified adenovirus is employed to treat head and neck cancer or neuroblastoma, the modified adenovirus may be administered to the vasculature at a site proximate to the head and neck cancer or neuroblastoma, whereby the modified adenovirus travels to and transduces the head and neck cancer cells or neuroblastoma cells. After the tumor cells or lymphoma cells are transduced with the modified adenovirus, an interaction agent or prodrug, such as, for example, ganciclovir, is administered to the patient, whereby the transduced tumor cells are killed.

In a further embodiment, the modified adenoviruses may be employed in the treatment of leukemias, including acute myeloid leukemia and acute lymphocytic leukemia. For example, the modified adenovirus may include a negative selective marker, or “suicide” gene, such as hereinabove described. The modified adenovirus may be administered intravascularly, or the modified adenovirus may be administered to the bone marrow, whereby the modified adenovirus transduces the leukemia cells. After the leukemia cells are transduced with the modified adenovirus, an interaction agent or prodrug is administered to the patient, whereby the transduced leukemia cells are killed.

In an alternative embodiment, leukemias, including acute myeloid leukemia and acute lymphocytic leukemia, or neuroblastoma, may be treated with a modified adenovirus including a DNA sequence encoding a polypeptide which elicits an immune response against the leukemia cells or neuroblastoma cells. Such polypeptides include, but are not limited to, immunostimulatory cyctokines such as Interleukin-2, co-stimulatory antigens, such as B7.1; and chemotactic agents, such as lymphotactin. When employed to treat leukemia, the modified adenovirus may be administered intravascularly, or may be administered to the bone marrow, whereby the modified adenovirus transduces the leukemia cells. When employed to treat neuroblastoma, the modified adenovirus may be administered directly to the neuroblastoma, and/or may be administered intravascularly, whereby the modified adenovirus transduces the neuroblastoma cells.

The transduced leukemia cells or the transduced neuroblastoma cells then express the polypeptide which elicits an immune response against the leukemia cells or the neuroblastoma cells, thereby inhibiting, preventing, or destroying the growth of the leukemia cells or neuroblastoma cells.

In yet another embodiment, the modified adenovirus may be employed to prevent or treat restenosis or prevent or treat vascular lesions after an invasive vascular procedure. The term “invasive vascular procedure,” as used herein, means any procedure that involves repair, removal, replacement, and/or redirection (e.g., bypass or shunt) of a portion of the vascular system, including, but not limited to arteries and veins. Such procedures include, but are not limited to, angioplasty, vascular grafts such as arterial grafts, removals of blood clots, removals of portions of arteries or veins, and coronary bypass surgery. For example, the modified adenovirus may include a DNA sequence encoding a therapeutic agent, such as cell cycle control agents, such as, for example, p21; hirudin; endothelial nitric oxide synthetase; or antagonists to cyclin G1 or cyclin D1, such as antibodies which recognize an epitope of cyclin G1 as cyclin D1. Alternatively, the modified adenovirus may include an antisense polynucleotide to the cyclin G1 or cyclin D1 gene, or in another alternative, the modified adenovirus may include a negative selective marker or “suicide” gene as hereinabove described. The modified adenovirus then is administered intravascularly, at a site proximate to the vascular lesion, or to the invasive vascular procedure, whereby the modified adenovirus transduces smooth muscle cells of the vasculature. The transduced cells then express the therapeutic agent, thereby treating or preventing restenosis or vascular lesions. Such restenosis or vascular lesions include, but are not limited to, restenosis or lesions of the coronary, carotid, femoral, or renal arteries, and renal dialysis fistulas.

In one embodiment, when the restenosis or vascular lesion is associated with proliferation of smooth muscle cells of the vasculature, the modified adenovirus may include a gene encoding a negative selective marker, or “suicide” gene as hereinabove described. Upon transduction of the smooth muscle cells with the modified adenovirus, an interaction agent or prodrug as hereinabove described is administered to the patient, thereby killing the transduced smooth muscle cells at the site of the restenosis or vascular lesion, and thereby treating the restenosis or vascular lesion.

In another embodiment, the modified adenovirus, which includes at least one DNA sequence encoding a therapeutic agent, may be administered to an animal in order to use such animal as a model for studying a disease or disorder and the treatment thereof. For example, a modified adenovirus, in accordance with the present invention, containing a DNA sequence encoding a therapeutic agent may be given to an animal which is deficient in such therapeutic agent. Subsequent to the administration of such modified adenovirus containing the DNA sequence encoding the therapeutic agent, the animal is evaluated for expression of such therapeutic agent. From the results of such a study, one then may determine how such adenoviruses may be administered to human patients for the treatment of the disease or disorder associated with the deficiency of the therapeutic agent.

It is also contemplated within the scope of the present invention that at least a portion, preferably at least a portion of the head portion, and more preferably the entire head portion, of the fiber of an adenovirus of a desired serotype may be incorporated into a gene delivery or gene transfer vehicle other than an adenovirus. Such gene delivery or gene transfer vehicles include, but are not limited to, viral vectors such as retroviral vectors, adeno-associated virus vectors, and Herpes virus vectors, such as Herpes Simplex Virus vectors; and non-viral gene delivery systems, including plasmid vectors, proteoliposomes encapsulating genetic material, “synthetic viruses,” and “synthetic vectors.”

When a viral vector is employed, the viral surface protein, such as a retroviral envelope, an adeno-associated virus naked protein coat, or a Herpes Virus envelope, is modified to include at least a portion, preferably at least a portion of the head portion, and more preferably the entire head portion, of an adenovirus of a desired serotype, whereby the viral vector may be employed to transduce cells having a receptor which binds to the head portion of the fiber of the adenovirus of the desired serotype. For example, the viral vector, which includes a polynucleotide (DNA or RNA) sequence to be transferred into a cell, may have a viral surface protein which has been modified to include the head portion of the fiber of Adenovirus 3. Such viral vectors may be constructed in accordance with genetic engineering techniques known to those skilled in the art. The viral vectors then may be employed to transduce cells, such as those hereinabove described, which include a receptor which binds to the head portion of the fiber of Adenovirus 3, to treat diseases or disorders such as those hereinabove described.

In another embodiment, the gene transfer vehicle may be a plasmid, to which is linked at least a portion, preferably at least a portion of the head portion, and more preferably the entire head portion, of the fiber of an adenovirus of a desired serotype. The at least a portion of the fiber of the adenovirus of a desired serotype may be bound directly to the plasmid vector including a polynucleotide to be transferred into a cell, or the at least a portion of the fiber of the adenovirus of a desired serotype may be attached to the plasmid vector by means of a linker moiety, such as, for example, linear and branched cationic polymers, such as, polyethyleneimine, or a polylysine conjugate, or a dendrimer polymer. The plasmid vector then is employed to transduce cells having a receptor which binds to the head portion of the fiber of the adenovirus of the desired serotype. For example, a plasmid vector may be attached, either through direct binding or through a linker moiety, to the head portion of the fiber of Adenovirus 3. The plasmid vector then may be employed to transduce cells having a receptor which binds to the head portion of the fiber of Adenovirus 3, as hereinabove described.

In another embodiment, a polynucleotide which is to be transferred into a cell may be encapsulated within a proteoliposome which includes at least a portion, preferably at least a portion of the head portion, and more preferably the entire head portion, of the fiber of an adenovirus of a desired serotype. The polynucleotide to be transferred to a cell may be a naked polynucleotide sequence or may be contained in an appropriate expression vehicle, such as a plasmid vector. The proteoliposome may be formed by means known to those skilled in the art. The proteoliposome, which encapsulates the polynucleotide sequence to be transferred to a cell, is employed in transferring the polynucleotide to cells having a receptor which binds to the head portion of the fiber of the adenovirus of a desired serotype. For example, the proteoliposome may include, in the wall of the proteoliposome, the head portion of the fiber of Adenovirus 3, and such proteoliposome may be employed in contacting cells, such as those hereinabove described, which include a receptor which binds to the head portion of the fiber of Adenovirus 3. Upon binding of the proteoliposome to the cell, the polynucleotide contained in the liposome is transferred to the cell.

In yet another embodiment, a polynucleotide which is to be transferred into the cell may be part of a “synthetic virus.” In such a “synthetic virus,” the polynucleotide is enclosed within an inner fusogenic layer of a pH sensitive membrane destabilizing polymer. The “synthetic virus” also includes an outer layer of a cleavable hydrophilic polymer. The at least a portion, preferably at least a portion of the head portion, and more preferably the entire head portion, of the fiber of an adenovirus of a desired serotype, is bound to the outer layer of the cleavable hydrophilic polymer. The polynucleotide to be transferred to a cell may be a naked polynucleotide sequence or may be contained in an appropriate expression vehicle as hereinabove described. The “synthetic virus” is employed in transferring the polynucleotide to cells having a receptor which binds to the head portion of the fiber of the adenovirus of a desired serotype. For example, the “synthetic virus” may include the head portion of the fiber of Adenovirus 3, which is bound to the cleavable hydrophilic polymer. The “synthetic virus” is employed in contacting cells which include a receptor which binds to the head portion of the fiber of Adenovirus 3. Upon binding of the “synthetic virus” to the cell, the polynucleotide contained in the “synthetic virus” is transferred to the cell.

In a further embodiment, a polynucleotide which is to be transferred into a cell may be part of a “synthetic vector”, wherein the polynucleotide is enclosed within a fusogenic layer of a fusogenic pH sensitive membrane destabilizing polymer. The at least a portion, preferably at least a portion of the head portion, and more preferably the entire head portion, of the fiber of an adenovirus of a desired serotype, is bound to the fusogenic pH sensitive membrane destabilizing polymer. Such a “synthetic vector” is useful especially for transferring polynucleotides to cells ex vivo or in vitro. For example, the “synthetic vector” may include the head portion of the fiber of Adenovirus 3, which is bound to the fusogenic pH sensitive membrane destabilizing polymer. The “synthetic vector” is employed in contacting cells which includes a receptor which binds to the head portion of the fiber of Adenovirus 3. Upon binding of the “synthetic vector” to the cell, the polynucleotide contained in the “synthetic vector” is transferred to the cell.

In accordance with yet another aspect of the present invention, there is provided an adenoviral vector of the Adenovirus 3 serotype which includes at least one heterologous DNA sequence. The at least one heterologous DNA sequence may be selected from those hereinabove described. Such adenoviral vectors may be employed in transducing cells, such as those hereinabove described, either in vivo, ex vivo, or in vitro, which include a receptor which binds to the head portion of the Adenovirus 3. The vectors may be administered in dosages such as those hereinabove described. The vectors may be administered in combination with a pharmaceutically acceptable carrier, such as those hereinabove described. Thus, such vectors may be employed to treat diseases or disorders such as those hereinabove described. It is to be understood, however, that the scope of this aspect of the present invention is not to be limited to the transduction of any particular cell type or the treatment of any particular disease or disorder.

Thus, in accordance with another aspect of the present invention, there is provided a method of transferring at least one polynucleotide into cells by contacting the cells with a gene transfer vehicle which includes at least a portion, preferably at least a portion of the head portion, and more preferably the entire head portion, of the fiber of Adenovirus 3. The cells include a receptor which binds to the at least a portion of the fiber of Adenovirus 3. Transfer of the at least one polynucleotide sequence into cells is effected through binding of the gene transfer vehicle to the cells. Such gene transfer vehicles include, but are not limited to, adenoviruses; retroviruses; adeno-associated virus; Herpes viruses such as Herpes Simplex Virus; plasmid vectors bound to the at least a portion, preferably the head portion, of the fiber of Adenovirus 3; and proteoliposomes encapsulating at least one polynucleotide to be transferred into cells. The at least one polynucleotide may encode at least one therapeutic agent such as those hereinabove described.

### EXAMPLES

The invention now will be described with respect to the following examples; however, the scope of the present invention is not intended to be limited thereby.

### Example 1

### Materials and Methods

Recombinant fiber plasmid. A shuttle plasmid was constructed for homologous recombination at the right hand end of Adenovirus 5 based adenoviral vectors. This shuttle plasmid, referred to as prepac, contains the last 8886 bp from 25171 bp to 34057 bp of the Ad dl327 (Thimmapaya, Cell, Vol. 31, pg. 543 (1983)) genome cloned into pBluescript SK II(+) (Stratagene) and was kindly supplied by Dr. Soumitra Roy, Genetic Therapy, Inc., Gaithersburg, Md. The wild type, Adenovirus 5 fiber cDNA contained within prepac was replaced with the 5TS3Ha cDNA using PCR gene overlap extension, as described in Horton, et al., Biotechniques, Vol. 8,pgs. 528-535 (1990). The 5TS3H contains the Adenovirus 5 fiber tail and shaft domains (5TS; amino acids 1 to 403) fused with the Adenovirus 3 fiber head region (3H, amino acids 136 to 319) as described in Stevenson, et al., J. Virol., Vol. 69, pgs. 2850-2857 (1995). The 5TS3Ha cDNA was modified to contain native 3′ downstream sequences of the wildtype 5F cDNA. In addition, the last two codons of the Adenovirus 3 fiber head domain, GAC TGA were mutated to correspond to the wild type, 5F codon sequence, GAA TAA to maintain the Adenovirus 5 fiber stop codon and polyadenylation signal. The Adenovirus 5 fiber 3′ downstream sequences were added onto the 5TS3Ha cDNA using the pgem5TS3H plasmid (Stevenson, 1995) as template and the following primers: P1:5′-CATCTGCAGCATGAAGCGCGCAAGACCGTCTGAAGATA-3′ (scs4) and P2:5′-CGTTGAAACATAACACAAACGAITCTTTATTCATCTTCTCTAATATAGGAAAAGGTAAk-3′ (scs 80). Overlapping homologous sequences were added onto prepac using the following primers: P3, 5′-TTACCTTTTCCTATATTAGAGAAGATGAATAAAGAATCGTTTGTGTTATGTTTCAACG-3′ (scs 79) and P4, 5′-AGACAAGCTTGCATGCCTGCAGGACGGAGC-3′ (scs81). Amplified products of the expected size were obtained and were gel purified. A second PCR reaction was carried out using the end primers, P1 and P4 to join the two fragments together. The DNA fragment generated in the second PCR reaction contained the 5TS3Ha cDNA with the last two codons mutated to the wildtype 5F sequence and the appropriate 3′ downstream prepac sequences. The 5TS3Ha PCR fragment was digested with NdeI and Sse8387 and was cloned directly into prepac to create the fiber shuttle plasmid, prep5TS3Ha.

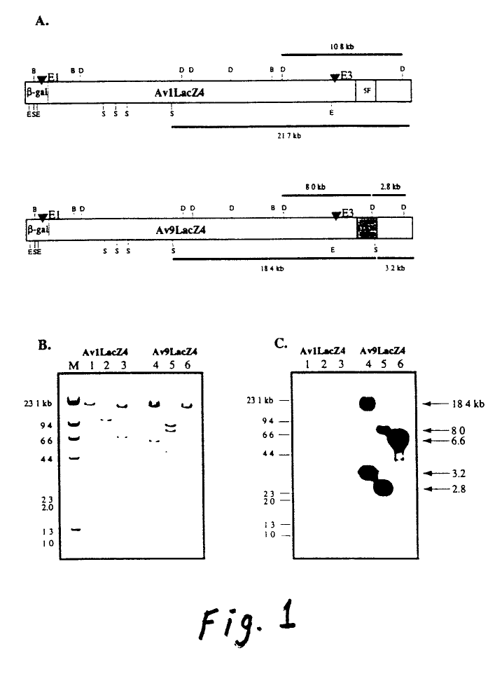

Generation of recombinant adenoviruses. The modified STS3Ha fiber cDNA was incorporated into the genome of Av1LacZ4, an E1 and E3-deleted adenoviral vector encoding β-galactosidase, and described in PCT Application No. WO95/09654, published Apr. 13, 1995, by homologous recombination between Av1LacZ4 and the prep5TS3Ha fiber shuttle plasmid to generate the chimeric fiber adenoviral vector referred to as Av9LacZ4. Human embryonic kidney 293 cells (ATCC CCL-1573) were obtained from the American Type Culture Collection (Rockville, Md.) and cultured in IMEM containing 10% heat inactivated FBS (HIFBS). Co-transfections of 293 cells were carried out with 10 μg of NotI-digested prep5TS3Ha and 1.5 μg of SrfI-digested Av1LacZ4 genomic DNA using a calcium phosphate mammalian transfection system (Promega Corporation, Madison, Wis.). The 293 cells were incubated with the calcium phosphate, DNA precipitate at 37° C. for 24 hours. The precipitate was removed and the monolayers were washed once with phosphate buffered saline (PBS). The transfected 293 cell monolayers were overlayered with 1% Sea Plaque agarose in MEM supplemented with 7.5% HIFBS, 2 mM glutamine, 50 units/ml penicillin, 50 μg/ml streptomycin sulfate, and 1% amphotericin B. Adenoviral plaques were isolated after approximately 14 days. Individual plaques were expanded, genomic DNA was isolated and screened for the presence of the chimeric fiber, 5TS3Ha cDNA using ScaI restriction enzyme digestion and confirmed by Southern blot analysis using the Ad3 fiber head as probe. Positive plaques were subjected to two rounds of plaque purification to remove parental, Av1LacZ4 contamination. The Av9LacZ4 vector after two rounds of plaque purification was expanded and purified by conventional techniques using CsCl ultracentrifugation. The adenovirus titers (particles/ml) were determined spectrophotometrically (Halbert, et al., J. Virol., Vol. 56, pgs. 250-257 (1985); Weiden, et al., Proc. Nat. Acad. Sci., Vol. 91, pgs. 153-157 (1994)) and compared with the biological titer (pfu/ml) determined using 293 cell monolayers as described in Mittereder, et al., J. Virol., Vol. 70, pgs. 7498-7509 (1996). The ratio of total particles to infectious particles (particles/pfu) was calculated. DNA was isolated from each vector and digested with DraI, ScaI, or EcoRI and BamHI to confirm the identity of each. The schematic diagrams of Av9LacZ4 and parental, Av1LacZ4 vectors are shown schematically in FIG. 1.

Expression of fiber constructs in baculovirus. As described previously (Stevenson, 1995), the baculovirus expression system (Clontech, Palo Alto, Calif.) was used to generate fiber proteins for receptor binding studies. Recombinant baculoviral vectors were used which expressed either the Ad5 fiber or Ad3 fiber proteins. Spodoptera frugiperda cells (Sf21) were cultured as monolayers at 27° C. in Grace's supplemented insect cell medium containing 10% HIFBS, 100 Units/ml penicillin, 100 μg/ml streptomycin sulfate, and 2.5 μg/ml of amphotericin B. Large scale infections of Sf21 cells with each recombinant fiber baculovirus were carried out and fiber containing cell lysates were prepared as described (Stevenson, 1995).

The Adenovirus 5 fiber protein was purified from the Sf21 cell lysates as described previously (Stevenson, 1995). Briefly, the Adenovirus 5 fiber trimer was purified to homogeneity using a two step purification procedure utilizing a DEAE-sepharose column, and then a Superose 6 gel filtration column equilibrated in PBS using an FPLC system (Pharmacia). Protein concentrations of the purified Adenovirus 5 fiber trimer and the insect cell lysates containing the Adenovirus 3 fiber (3F/CL) were determined by the bicinchoninic acid protein assay (Pierce, Rockford, Ill.) with bovine serum albumin (BSA) as the assay standard.

The expression of fiber proteins was verified by sodium dodecyl sulfate (SDS)-4/15% polyacrylamide gel electrophoresis (PAGE) under denaturing conditions and Western immunoblot analysis. The proteins were transferred to a polyvinylidene difluoride (PVDF) membrane by use of a small transblot apparatus (Biorad, Hercules, Calif.) for 30 minutes at 100 volts. After the transfer was completed, the PVDF membrane was stained transiently with Ponceau red and the molecular weight standards were marked directly on the membrane. Molecular weight standards used ranged from 200 to 14 kDa (Biorad). Nonspecific protein binding sites on the PVDF membrane were blocked using a 5% dried milk solution in 10 mM Tris, pH7.4 containing 150 mM NaCl, 2 mM EDTA 0.04% Tween-20 for one hour at room temperature or overnight at 4° C. The membrane then was incubated for one hour at room temperature with a 1:10,000 dilution of the primary anti-Adenovirus 2 fiber monoclonal antibody, 4D2-5 (ascites kindly provided by Dr. J. Engler, University of Alabama) or with 70 μg/ml of a partially purified anti-Adenovirus 3 fiber specific rabbit polyclonal antibody generated against the baculoviral expressed Adenovirus 3 fiber head domain (Stevenson, 1995). The membrane was developed with either a 1:10,000 dilution of the secondary sheep anti-mouse IgG horseradish peroxidase (HRPO)-conjugated antibody (Amersham Lifesciences, Arlington, Ill.) or with a 1:2000 dilution of donkey anti-rabbit IgG-HRPO using an enhanced chemiluminescence system (Amersham Lifesciences). The membrane was exposed to film for approximately 3 to 60 seconds.

Production of an anti-Adenovirus 3 fiber specific antiserum. The fiber head region of the Adenovirus 3 fiber was expressed using the baculoviral expression system as described (Stevenson, 1995). The insect cell lysate containing the Adenovirus 3 fiber head was used for immunizations of New Zealand White rabbits according to standard protocols (Lofstrand Labs Ltd, Gaithersburg, Md.). The IgG fraction was isolated and was applied to an affinity column containing covalently bound insect cell lysate proteins. The unbound fraction from this affinity column was obtained and tested for immunoreactivity against the Adenovirus 5, Adenovirus 3, and chimeric, 5TS3H fiber proteins using Western blot analysis.

Competitive viral transduction assay. The receptor tropism of the recombinant adenoviruses was evaluated using a viral transduction assay in the presence of fiber protein competitors. Monolayers of HeLa cells (ATCC CCL 2) cultured in DMEM with 10% HIFBS, 100 Units/ml penicillin, and 100 μg/ml streptomycin sulfate contained in 12 well dishes were incubated with various dilutions of either purified Adenovirus 5 fiber trimer protein (0.05 μg/ml up to 100 μg/ml) or with an insect cell lysate containing the Adenovirus 3 fiber (100 μg/ml up to 2000 μg/ml) for one hour at 37° C. in a total volume of 0.2 ml of DMEM, 2% HIFBS. The chimeric fiber Av9LacZ4 or parental, Av1LacZ4 adenoviral vectors were then added in a total volume of 5 μl to achieve a total particle per cell ratio of 100 by dilution of the virus into DMEM, 2% HIFBS. Virus transductions were carried out for 1 hour at 37° C. The monolayers were washed once with PBS, 1 ml of DMEM, 109 HIFBS was added per well, and the cells were incubated for an additional 24 hours to allow for β-galactosidase expression. The cell monolayers then were fixed using 0.56 glutaraldehyde in PBS and then were incubated with 1 mg/ml 5-bromo-4-chloro-3-indolyl-b-D-galactoside (X-gal), 5 mM potassium ferrocyanide, 2 mM MgCl₂ in 0.5 ml PBS. The cells were stained approximately 24 hours at 37° C. The monolayers were washed with PBS and the average number of blue cells per high power field were quantitated by light microscopy using a Zeiss ID03 microscope, three to five fields were counted per well. The average number of blue cells per high power field was expressed as a percentage of the control which did not contain competitor fiber protein. Each concentration of competitor was carried out in triplicate and the average percentage ± standard deviation was expressed as a function of added competitor fiber protein. Each experiment was carried out three to four times and data from a representative experiment is shown.

Cell Culture. The transduction efficiency of Av9LacZ4 and Av1LacZ4 was surveyed on a panel of human cell lines. HeLa, MRC-5 (ATCC CCL-171), FaDu (ATCC HTB 43), and THP-1 (ATCC TIB-202) cells were obtained from the ATCC and cultured in the recommended medium. Human umbilical vein endothelial cells (HUVEC, CC-2517) and coronary artery endothelial cells (HCAEC, CC-2585) were obtained from the Clonetics Corporation (San Diego, Calif.) and cultured in the recommended medium. Each cell line was transduced with the chimeric fiber Av9LacZ4 or the wild type, Av1LacZ4 adenoviral vectors at 0, 10, 100, and 1000 total particles per cell for one hour at 37° C. in a total volume of 0.2 ml of culture medium containing 2% HIFBS. The cell monolayers were then washed once with PBS and 1 ml of the appropriate culture medium containing 10% HIFBS was added. THP-1 cells were incubated with the indicated concentration of vector for one hour at 37° C. in a total volume of 0.2 ml of culture medium containing 2% HIFBS, and then 1 ml of complete medium containing 10% HIFBS was added. The cells were incubated for 24 hours to allow for β-galactosidase expression. The cell monolayers were then fixed and stained with X-gal as described above. The incubation of each cell line in the X-gal solution varied from 1.5 hours up to 24 hours depending on the amount background staining found in the mock infected wells. The percent transduction was determined by light microscopy by counting the number of transduced, blue cells per total cells in a high power field using a Zeiss ID03 microscope, three to five fields were counted per well. Each vector dose was carried out in triplicate and the average percent transduction per high power field (mean ±sd, n=3 wells) was determined and expressed as a function of added vector. Each cell line was transduced at least three times and the data represents the mean percent transduction ± standard deviation from three independent experiments.

### Results

Construction of an adenovirus vector containing a chimeric fiber gene. It was shown previously using chimeric fiber proteins expressed in vitro and in insect cells that the receptor specificity of the adenovirus fiber protein can be altered by exchanging the head domain with another serotype which recognizes a different receptor (Stevenson, 1995). To generate an adenoviral vector particle with an altered receptor specificity, the chimeric fiber gene containing the Adenovirus 3 fiber head domain fused to the Adenovirus 5 fiber tail and shaft, 5TS3H, was incorporated within the adenoviral genome of Av1LacZ4. For the precise replacement of the wild type Adenovirus 5 fiber gene, a shuttle plasmid was constructed which contained the last 8886 bp of the Ad dl327 genome from 73.9 to 100 map units including the Adenovirus 5 fiber gene, E4 and the right ITR. This shuttle plasmid was used for incorporation of modified fiber genes into the backbone of an E1 and E3 deleted adenoviral vector Av1LacZ4 via homologous recombination. This strategy replaces the native Adenovirus 5 fiber with the chimeric 5TS3H fiber sequences cloned within the prep5TS3Ha shuttle plasmid. The resulting vector, Av9LacZ4 contains the nuclear targeted β-galactosidase cDNA and the Adenovirus 3 fiber head domain. This approach will allow for any modification to the native fiber sequence to be incorporated within the adenoviral genome.

Both the parental, Av1LacZ4 and the chimeric fiber Av9LacZ4 vectors are shown schematically in FIG. 1. The Adenovirus 3 fiber head region introduces additional DraI and ScaI restriction enzyme sites within the Av1LacZ4 genome which were used to identify the recombinant virus. Plaques which yielded the predicted DraI and ScaI diagnostic fragments as indicated in FIG. 1A were selected and expanded. Genomic DNA isolated from the purified chimeric fiber, Av9LacZ4 and the parental, Av1LacZ4 viruses was analyzed by restriction enzyme digestion and agarose gel electrophoresis (FIG. 1B). The expected DNA fragments were obtained for both the Av9LacZ4 and wild type, Av1LacZ4 viruses. Diagnostic 18.4 and 3.2 kb fragments were found after ScaI digestion of the Av9LacZ4 genomic DNA (FIG. 1B, lane 4) indicating the presence of the Adenovirus 3 fiber head domain. DraI restriction endonuclease digestion of Av9LacZ4 also confirmed the presence of the Adenovirus 3 fiber head domain as indicated by the 8.0 and 2.8 kb diagnostic fragments (FIG. 1B, lane 5). EcoRI and BamHI digestion produced an identical restriction pattern for both vectors as expected (FIG. 1B, lanes 3 and 6). Southern blot analysis using the Adenovirus 3 fiber head probe demonstrated the expected hybridization pattern for all restriction endonuclease digestions for both vectors (FIG. 1C). The 18.4 and 3.2 kb ScaI and the 8.0 and 2.8 kb DraI diagnostic fragments of Av9LacZ4 hybridized with the Adenovirus 3 fiber head probe (FIG. 1C, lanes 4 and 5). The 6.6 kb EcoRI/BamHI fragment which contains the full length 5TS3H fiber gene was also detected (FIG. 1C, lane 6). Southern blot analysis using the Adenovirus 5 fiber head probe (data not shown) demonstrated the expected hybridization pattern for Av1LacZ4 and confirmed that the chimeric fiber Av9LacZ4 virus preparation was free of parental, Av1LacZ4 virus.

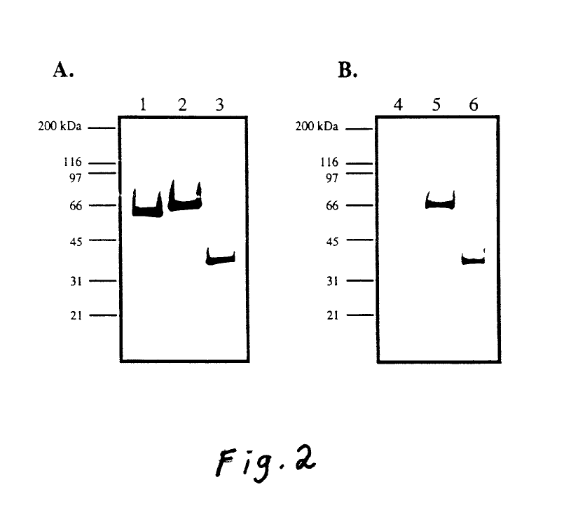

Characterization of adenoviral particles containing the chimeric fiber. Expression and assembly of the chimeric 5TS3H fiber protein into the adenoviral capsid was examined by Western Blot analysis of CsCl purified virus stocks. An equivalent number of the parental (Av1LacZ4) and chimeric (Av9LacZ4) particles were subjected to 4/1596 SDS PAGE under denaturing conditions. A control virus containing a full length Ad3 fiber was also analyzed. Western immunoblot analysis was carried out using an anti-fiber monoclonal antibody, 4D2-5 (FIG. 2A) and a rabbit polyclonal antibody specific for the Ad3 fiber head domain (FIG. 2B). The 4D2-5 antibody recognizes a conserved epitope located within the N-terminal tail domain of the fiber protein (Hong, et al., Embo. J., Vol. 14, pgs. 4714-4727 (1995)) and reacts with both the Adenovirus 5 (5F) and the Adenovirus 3 (3F) fiber proteins (Stevenson, 1995). As shown in FIG. 2A, the Av1LacZ4 (lane 1) and Av9LacZ4 (lane 2) viruses contain fiber proteins of approximately 62 to 63 kDa which react with the 4D2-5 antibody while the Adenovirus 3 fiber virus contains a fiber protein of approximately 35 kDa (FIG. 2A, lane 3). The presence of the Adenovirus 3 fiber head (3FH) domain within the 5TS3H chimeric fiber was confirmed by Western Blot analysis using a rabbit polyclonal antibody specific for the Adenovirus 3 fiber. The rabbit anti-3FH polyclonal antibody did not bind to the Adenovirus 5 fiber protein in Av1LacZ4 and was specific for the 35 kDa, Adenovirus 3 fiber protein in the control virus (FIG. 2B, lane 6) and the Adenovirus is fiber head domain contained within the chimeric 5TS3H fiber protein in Av9LacZ4 (FIG. 2B, lane 5).

The biological titers and particle numbers of the chimeric fiber (Av9LacZ4) and parental (Av1LacZ4) adenoviruses were compared. Biological titers determined using 293 cell monolayers indicated plaque forming titers of 2.6 and 4.5×10¹⁰ pfu/ml for the Av1LacZ4 and Av9LacZ4 viral preparations, respectively. The total particle concentrations were determined spectrophotometrically and were 1.45 and 1.25 ×10¹² particles/ml for Av1LacZ4 and Av9LacZ4, respectively. Thus, the ratio of particle number to pfu titer was similar for both viruses, 55.8 versus 27.8 total particles/pfu, respectively. An increased ratio of particle number to infectious titer has previously been reported for Adenovirus 3 compared to Adenovirus 2 (Defer, et al., J. Virol., Vol. 64, pgs. 3661-3673 (1990)); however, the replacement of the Adenovirus 5 fiber head domain with the Adenovirus 3 fiber head domain did not adversely affect the cellular production of the adenovirus containing the chimeric fiber protein or significantly change the ratio of total physical to infectious particles.

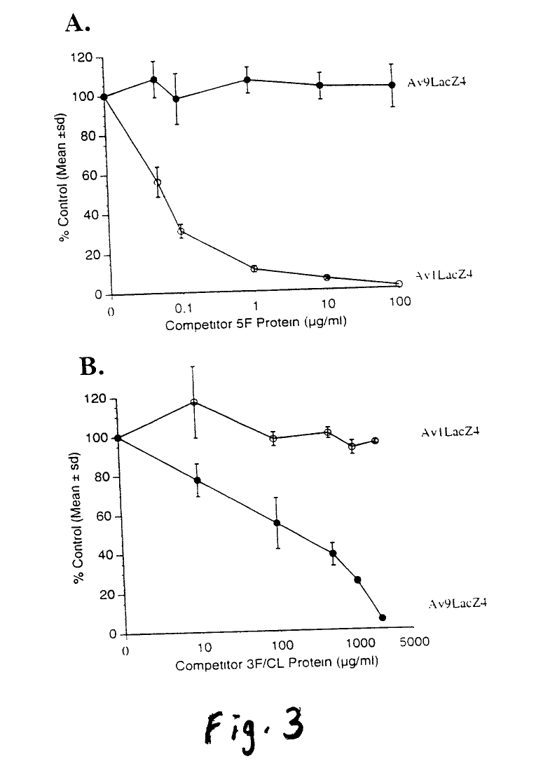

Receptor binding specificity of the modified fiber adenovirus. To evaluate the receptor binding properties of the chimeric fiber vector compared to the native Adenovirus 5 fiber vector, transduction experiments were carried out in the presence of recombinant fiber protein competitors. Cells were preincubated with purified Adenovirus 5 fiber protein or with an insect cell lysate containing the Adenovirus 3 fiber protein prior to transduction with the chimeric fiber or native Adenovirus 5 fiber vector. FIG. 3 shows the results of transduction experiment3 in which HeLa cells were incubated with increasing amounts of Adenovirus 5 fiber protein (FIG. 3A) or with the Adenovirus 3 fiber competitor (FIG. 3B) prior to transduction with the Av9LacZ4 or Av1LacZ4 vectors. Transduction of HeLa cells with Av1LacZ4 decreased with increasing amounts of Adenovirus 5 fiber trimer protein, with maximal competition occurring between 0.1 to 1.0 μg/ml. In contrast, the purified Adenovirus 5 fiber trimer did not block the transduction of the Av9LacZ4 chimeric fiber adenovirus. These results confirm that the wild type, Av1LacZ4 and Av9LacZ4 chimeric fiber vectors bind to different cell surface receptors. This conclusion was supported by the reciprocal experiment shown in FIG. 3B. Increasing concentrations of the Adenovirus 3 fiber competitor decreased the AV9LacZ4 transduction of HeLa cells but did not influence transduction with the wild type, Av1LacZ4 vector. The competition between the Adenovirus 3 fiber competitor and Av9LacZ4 was specific since control experiments carried out with insect cell lysates which did not contain the Adenovirus 3 fiber protein did not result in competition (data not shown). These results indicate that transduction of HeLa cells by Av9LacZ4 is mediated by the chimeric fiber protein which interacts with the Adenovirus 3 receptor. Thus, the modification of the Adenovirus 5 fiber head domain has resulted in a change in receptor tropism of an adenoviral vector.

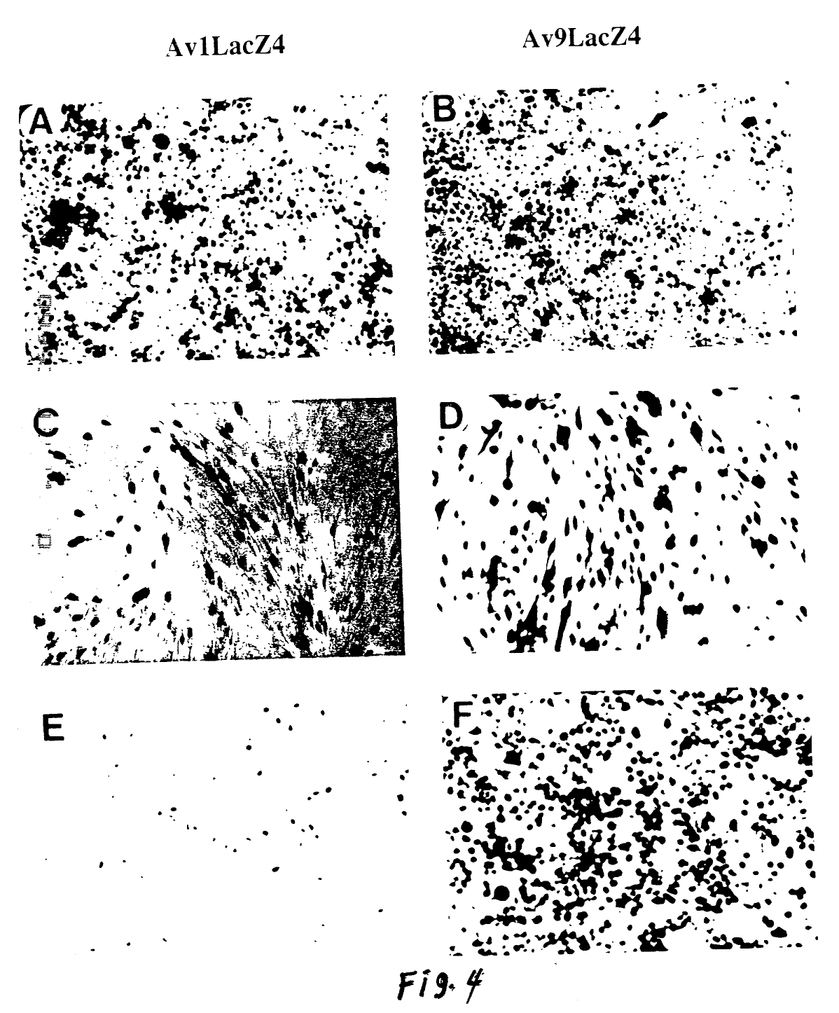

Transduction of human cell lines by the chimeric fiber vector. Because the identity of the Adenovirus 5 and Adenovirus 3 receptors is unknown, there is relatively little information available concerning their cellular distribution. It was hypothesized that differential expression of the Adenovirus 5 and Adenovirus 3 receptors on different human cells might be reflected in the differential transduction by the parental, Av1LacZ4 and chimeric fiber, Av9LacZ4 vectors. The transduction properties of a number of human cell lines by the two vectors was investigated. Several cell lines were included which had been identified as negative for Adenovirus 5 fiber adenovirus receptor binding (Haung, et al., J. Virol., Vol. 70, pgs. 4502-4508 (1996); Stevenson, 1995) and/or refractory to Av1LacZ4 infection (unpublished data). Cells ware infected with the chimeric fiber, Av9LacZ4 or the wild type, Av1LacZ4 adenovirus at particle per cell ratios of 0, 10, 100, and 1000 in a total volume of 0.2 ml of culture medium. 24 hours later the cells were stained with X-gal as hereinabove described. Shown in FIG. 4 are representative photographs of the Av1LacZ4 and Av9LacZ4 transduction of HeLa cells (FIGS. 4A and 4B), MRC-5, a human embryonic lung fibroblast cell line (FIGS. 4C and 4D), and FaDu, a human squamous cell carcinoma line (FIGS. 4E and 4F) monolayers at the 1000 virus particles per cell dose. Both vectors transduced HeLa cell monolayers with similar efficiencies. In contrast, differential transduction of the MRC-5 and FaDu cell lines was found. Both the MRC-5 and FaDu cells were relatively refractory to Av1LacZ4 transduction but were readily transduced with Av9LacZ4.

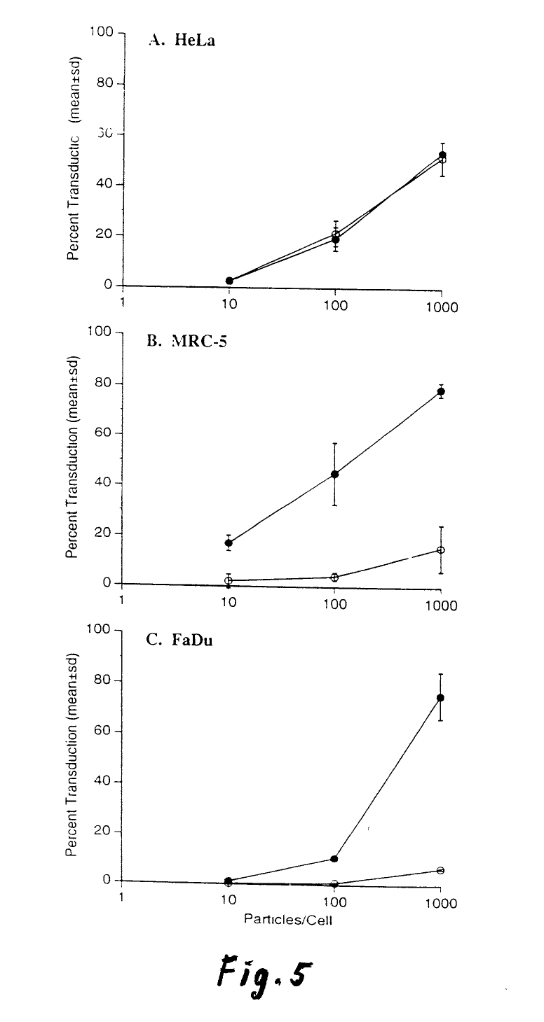

The percent transduction of each cell line was quantitated and the fraction of HeLa, MRC-5, and FaDu cells transduced as a function of dose is shown in FIG. 5. HeLa cells (FIG. 5A) were equally susceptible to transduction with both vectors indicating that both the Adenovirus 5 and Adenovirus 3 receptors are present on the cell surface. The MRC-5 (FIG. 5B) human embryonic lung cell line was efficiently transduced with the chimeric fiber, Av9LacZ4 vector. The percent transduction with Av9LacZ4 was dose dependent with approximately 80% transduction at the vector dose of 1000. Less efficient transduction of MRC-5 cells with Av1LacZ4 was observed suggesting that these cells either lack or express low levels of the Adenovirus 5 receptor. In contrast, the Adenovirus 3 receptor appears to be abundant on this cell type. The FaDu cell monolayers (FIG. 5C) were also transduced more efficiently with Av9LacZ4 with 75% of the cells transduced at the vector dose of 1000 compared to only 7% transduction achieved with Av1LacZ4 at the same vector dose.

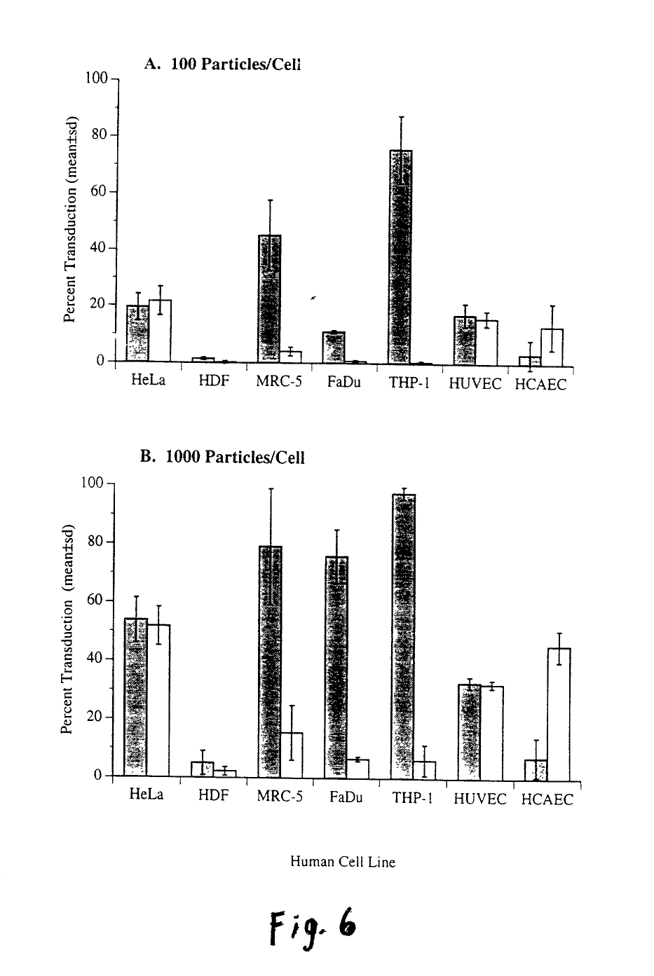

The transduction of a number of additional human cell lines were compared using Av1LacZ4 and Av9LacZ4. FIG. 6 summarizes data for each of the cell lines examined at the virus particle per cell ratios of 100 (FIG. 6A) and 1000 (FIG. 6B). The cell lines assessed in addition to the HeLa, MRC-5, and FaDu cell lines included HDF, human diploid fibroblasts; THP-1, human monocytes; HUVEC, human umbilical vein endothelial cells; and HCAEC, human coronary artery endothelial cells. Cells were infected with Av9LacZ4 or Av1LacZ4 adenoviral vectors at particle per cell ratios of 100 and 1000 and 24 hours later were stained with X-gal as hereinabove described. The fraction of transduced cells for each cell line at the indicated vector dose was determined. As shown previously, Hela cells were transduced at equivalent levels using both adenoviral vectors, while HDF cells were refractory to Av1LacZ4 as well as Av9LacZ4 transduction. HDF cells are negative for Adenovirus 5 fiber binding indicating that these cells lack or express low levels of the Adenovirus 5 receptor (Stevenson, 1995). The transduction data presented in FIG. 6 for HDF cells suggests that these cells lack or express low levels of the Adenovirus 3 receptor as well.

This analysis identified several human cell lines which were transduced differentially by the parental, Av1LacZ4 and the chimeric fiber, Av9LacZ4 vectors. MRC-5, FaDu, and THP-1 cells were efficiently infected with the recombinant vector containing the Adenovirus 3 fiber head in a dose dependent manner (FIGS. 6A and 6B), suggesting that the Adenovirus 3 receptor is more abundant than the Adenovirus 5 receptor on these cell types. At the vector dose of 1000 particles per cell approximately 450 of the HCAEC cells were transduced with the wild type fiber, Av1LacZ4 vector while only 7.3% were transduced with the chimeric fiber Av9LacZ4 vector. Venous endothelial cells (HUVEC) were equivalently transduced with both vectors. Differences in transduction of arterial and venous endothelial cells with Av1LacZ4 and Av9LacZ4 reveals the differential expression of the Adenovirus 3 and Adenovirus 5 receptors on cells derived from different regions of the vasculature. These data taken together demonstrate the differential expression of the Adenovirus 5 and Adenovirus 3 receptors on human cell lines derived from target tissues which are of potential clinical relevance.

### Discussion

A major goal in gene therapy research is the development of vectors and delivery systems which can achieve efficient targeted in vivo gene transfer and expression. Vectors are needed which maximize the efficiency and selectivity of gene transfer to the appropriate cell type for expression of the therapeutic gene and which minimize gene transfer to other cells or sites in the body which could result in toxicity or unwanted side effects. Of the viral vectors under investigation for in vivo gene transfer applications, the adenovirus system has shown considerable promise and has undergone extensive evaluation in animal models as well as early clinical evaluation in lung disease and cancer. A key feature of adenovirus vectors is the efficiency of transduction and the resulting high levels of gene expression which can be achieved in vivo. This is derived from the ability to prepare high titer stocks of purified vector and from the remarkable efficiency of each of the steps in the adenoviral entry process leading to gene expression (Greber, et al., Cell, Vol. 75, pgs. 477-486 (1993)). Attachment of adenovirus particles to the cell is mediated by a high affinity interaction between the fiber protein and the cellular receptor (Philipson, et al. J. Virol., Vol. 2, pgs. 1064-1075 (1968)). Following binding, virion entry into many cell types is facilitated by an interaction between RGD peptide sequences in the penton base and the αvb3 and αvb5 integrins which act as co-receptors (Wickham, et al., Cell, Vol. 73, pgs. 303-319 (1993)). In the absence of the high affinity interaction of the fiber protein with its receptor, viral binding and transduction can still occur but with reduced efficiency. This fiber independent binding and transduction is believed to occur via a direct association between the penton base and cellular integrins (Haung, 1996). As the first step in the cellular transduction process, the interaction between the fiber protein and the cell is an attractive and logical target for controlling the cell specificity of transduction by adenoviral vectors. It has been shown that the receptor binding domain of the fiber protein resides within the trimeric globular head domain (Henry, et al., J. Virol., Vol. 68, pgs. 5239-5246 (1994); Louis, et al., J. Virol., Vol. 68, pgs. 4104-4106 (1994); Stevenson, 1995). The interaction of the fiber head domain with its receptor thus determines the binding specificity of adenoviruses. Consequently, manipulation of the fiber head domain represents an opportunity for control of the cell specificity of transduction by adenovirus vectors.

In order to test this concept experimentally, advantage was taken of the fact that adenoviruses of the group B and group C serotypes bind to different cellular receptors (Defer, 1990; Mathias, et al., J. Virol., Vol. 68, pgs. 6811-6814 (1994); Stevenson, 1995). Chimeric fiber proteins were prepared which exchanged the head domains of the Adenovirus 3 and Adenovirus 5 fiber proteins. Cell binding and competition studies with the recombinant chimeric fiber proteins confirmed the role of the fiber head domain in receptor binding and showed that an exchange of head domains resulted in a corresponding change of receptor specificity between the Adenovirus 3 and Adenovirus 5 receptors (Stevenson, 1995). In the present study, we have extended this analysis by the construction of an Adenovirus 5 based adenoviral vector, Av9LacZ4 which contains the fiber head domain from Adenovirus 3. The fiber modified vector was prepared by a gene replacement strategy using the β-galactosidase expressing vector Av1LacZ4 as a starting point. A plasmid cassette containing the Adenovirus 5/Adenovirus 3 chimeric fiber gene, 5TS3H was used for homologous recombination with the Av1LacZ4 genome resulting in the precise substitution of the Adenovirus 5 fiber gene with the chimeric fiber gene containing the Adenovirus 3 fiber head to generate Av9LacZ4. Following plaque purification, molecular analysis of the recombinant vector genome provided confirmation of the fiber gene replacement in the vector. Western Blot analysis of purified vector particles using an antiserum specific for the Adenovirus 3 fiber verified the expression and assembly of the chimeric, 5TS3H fiber protein into functional adenoviral particles. The changed receptor specificity of the Av9LacZ4 chimeric fiber vector was confirmed by competition with recombinant fiber proteins which showed that transduction of 293 cells was effectively blocked by soluble Adenovirus 3 fiber but not by Adenovirus 5 fiber. This data confirms previous results obtained from binding experiments with recombinant fiber proteins and extends the analysis to intact adenovirus particles. Furthermore, the changed receptor specificity of the Av9LacZ4 vector establishes experimentally that the tropism of adenovirus vectors can be altered by manipulating the head domain.

The titer, yield, and ratio of physical to infectious particles of the fiber chimeric vector Av9LacZ4 and the parental Adenovirus 5, Av1LacZ4 vector were similar, thus indicating that the fiber head exchange did not alter significantly the growth properties of the vector on 293 cells. It has been reported that the infectivity of Adenovirus 3 is significantly less than that of Adenovirus 5, with Adenovirus 3 having a particle to PFU ratio approximately 20 times that of Adenovirus 5 (Defer, 1990). The similar infectivity of the Av9LacZ4 vector to the parental, Av1LacZ4 vector shows that the efficiency of entry of an Adenovirus 5 based vector via either the Adenovirus 5 or Adenovirus 3 receptor is similar. This suggests that the differences in the infectivity between Adenovirus 5 and Adenovirus 3 are not due to the use of a different receptor for binding and must reflect other differences between the two serotypes. The finding that the infectivity of the Av1LacZ4 and Av9LacZ4 vectors in 293 cells is similar leads to the important conclusion that the binding specificity of adenovirus vectors can be completely changed without affecting adversely the subsequent steps in entry and disassembly of the vector particles leading to nuclear gene delivery and expression. The implication of this result is that the function of the fiber receptor is primarily to promote efficient cellular attachment and that cell entry is an independent event which is not necessarily dependent on the molecule used for attachment. Therefore, it should be possible to modify the fiber protein to promote vector attachment to a range of different cell surface molecules without compromising the ability of the vector to enter the cell. This conclusion is supported by a recent report of a fiber modified adenovirus which binds to ubiquitously expressed cell surface proteoglycans and as a result has an extended cell tropism (Wickham, et al., Nature Biotechnology, Vol. 14, pgs. 1570-1573 (1996)). It should therefore be possible to construct other adenovirus vectors containing fiber proteins modified to contain ligands for cellular receptors which are expressed in a cell specific manner and as a result to achieve cell selective transduction.

The importance of the interaction between the fiber protein and the cellular fiber receptor for adenovirus infectivity is underscored by the fact that blockade of this interaction by soluble fiber protein results in the efficient inhibition of transduction (FIG. 3). Furthermore, cells which lack or express low levels of the cellular fiber receptor are inefficiently transduced and high levels of input vector are needed to achieve gene transfer (Haung, 1996). Recent clinical experience with adenoviral vectors in the treatment of cystic fibrosis lung disease has revealed a previously unsuspected resistance of human airway cells to transduction by Adenovirus 5 based vectors (Grub, et al., Nature, Vol. 371, pgs. 802-806 (1994); Zabner, et al. J. Virol., Vol. 70, pgs. 6994-7003 (1996)). It has been proposed that patterns of expression of both the αv integrins and the fiber attachment receptors may be involved in limiting transduction of human airway in vivo (Goldman, et al., J. Virol., Vol. 69, pgs. 5951-5958 (1995); Zabner, 1996). Evidence for a correlation between the level of αv integrin expression on human pulmonary epithelial cells and the efficiency of adenoviral vector transduction supports this hypothesis (Goldman, 1995).

The distribution of the Adenovirus 5 fiber attachment receptor on primary human cells is poorly characterized, largely due to the fact that its identity is unknown; however, it is increasingly clear that many human cell lines and a number of primary cells are refractory to transduction by Adenovirus 5 based vectors due to low levels or absence of the Adenovirus 5 fiber receptor. As noted previously, the Adenovirus 3 fiber receptor, while also as yet unknown, is clearly distinct from the Adenovirus 5 fiber receptor. Consequently, if differences in the pattern of expression of the two receptors exist, this should be reflected in a differential transduction efficiency by vectors which attach to either the Adenovirus 5 or Adenovirus 3 fiber receptors. In support of this hypothesis, several human cell lines have been identified, which were inefficiently transduced by the Adenovirus 5 vector, Av1LacZ4 and which could be transduced more efficiently by the chimeric fiber, Av9LacZ4 vector. These include a human head and neck tumor line FaDu, a human lung epithelial cell line MRC-5, and a human monocytic cell line THP-1. Transduction of HeLa cells and human umbilical vein endothelial cells (HUVEC) was equally efficient with both vectors. In contrast, human coronary artery endothelial cells (HCAEC) were more efficiently transduced by the Av1LacZ4 than by Av9LacZ4. Because the only difference between the two vectors is the identity of the fiber head domain, the differences observed in transduction are fiber dependent and must be a result of the differential expression of the two fiber receptors. The overlapping but distinct cellular distribution of the fiber receptors for Adenovirus 5 and Adenovirus 3 which is revealed by these results will likely be of practical value in designing vectors for transduction of specific human target cells. For example, the results obtained with the THP-1 cell line suggests that gene transfer to the monocyte/macrophage linage will be more efficient with vectors having the Adenovirus 3 receptor tropism than that of Adenovirus 5. Previous studies have demonstrated that human hematopoietic cells, monocytes, T-lymphocytes, and THP-1 cells were refractory to adenoviral vector transduction due to an apparent lack of Adenovirus 5 fiber receptors and were transduced only at high doses of input Adenovirus 5 vector (Haung, et al., J. Virol., Vol. 64, pgs. 2257-2263 (1995); Haung, 1996). The efficient transduction of monocytes with the Av9LacZ4 vector suggests that it may be useful in designing strategies for the treatment of cardiovascular disease and atherosclerosis by targeting macrophage cells in vessel wall lesions. Similarly, the FaDu cell data indicates that certain tumor cells will be transduced more effectively with the Av9LacZ4 vector than with Av1LacZ4.

The ability to modify adenoviral vectors to improve or enable transduction will increase the efficiency of adenoviral-mediated gene transfer. Modifications to the adenoviral fiber protein such as the head replacement strategy described in the present study is an approach which can lead to highly selective transduction of target cells. Head domains from other fiber proteins can be used to construct chimeric fibers which target vectors to alternative adenoviral receptors exploiting natural differences in the tropism of different adenoviral serotypes. Novel fiber proteins can also be constructed by replacement of the fiber head domain with other trimeric proteins, fusion of peptide sequences onto the Adenovirus 5 fiber C-terminus (Michael, et al., Gene Ther., Vol. 2, pgs. 660-668 (1995)) or addition of peptide ligands within exposed loop regions of the fiber head domain (Xia, et al., Structure, Vol. 2, pgs. 1259-1270 (1994)). These strategies will lead to the development of customized adenoviral vectors which selectively target specific cell types.

### Example 2

### Transduction of Lung Carcinoma Cell Lines

The A549 lung carcinoma (ATCC No. CCL-185), H23 lung adenocarcinoma (ATCC No. CRL-5800), H358 lung bronchiolalveolar carcinoma (ATCC No. CRL-5807), H441 lung papillary adenocarcinoma (ATCC No. HTB-174), and H460 lung large cell carcinoma cell lines (ATCC No. HTB-177) were transduced with Av1LacZ4 or Av9LacZ4 at 100 or 1,000 particles per cell according to the procedure of Example 1. Transduction data are given in Table I below.

TABLE I

| | Av9LacZ4 | | Av1LacZ4 | |
|---|---|---|---|---|
| | particles/cell | | particles/cell | |
| Cell Line | 100 | 1,000 | 100 | 1,000 |
| A549 | ++ | ++++ | −/+ | +++ |
| H23 | +++ | +++ | +++ | +++ |
| H358 | +++ | ++++ | −/+ | ++ |
| H441 | ++ | ++++ | −/+ | −/+ |
| H460 | +++ | ++++ | ++ | +++ |

−/+ 0-25% transduction
++ 25-50% transduction
+++ 50-75% transduction
++++ 75-100% transduction

The above data suggests that an adenoviral vector having a head portion from an Adenovirus 3 fiber can be employed for the transduction of lung carcinoma cells, and for the treatment of lung cancer.

### Example 3

Transduction of Lymphoma and Leukemia Cells

U937 human histiocytic lymphoma cells (ATCC CRL-1593) were transduced with Av1LacZ4 or Av9LacZ4 at 100 or 1,000 particles/cell as described hereinabove in Example 1. Each experiment was carried out in triplicate, and the mean percentage of transduced cells was determined. No transduction was observed of U937 cells contacted with Av1LacZ4 at 100 particles/cell, and only 0.1% transduction of U937 cells was observed at 1,000 Av1LacZ4 particles/cell. In contrast, there was 3.4% ±1.0% transduction of U937 cells with Av9LacZ4 at 100 particles/cell, and 9.2% ±0.4% transduction of U937 cells with Av9LacZ4 at 1,000 particles/cell.

In another experiment, K562 human chronic myelogenous leukemia cells (ATCC CCL243) were transduced with Av1LacZ4 or Av9LacZ4 at a multiplicity of infection (MOI) of 10, 50, or 100 according to the procedure of Example 1. Transduction results are given in Table II below.

TABLE II

| Av9LacZ4 | | Av1LacZ4 | |
|---|---|---|---|
| MOI | | MOI | |
| 10 | ++ | 10 | −/+ |
| 50 | ++++ | 50 | ++ |
| 100 | ++++ | 100 | +++ |

−/+ 0-25% transduction
++ 25-50% transduction
+++ 50-75% transduction
++++ 75-100% transduction

In another experiment, KG1 human bone marrow, acute myelogenous leukemia cells (ATCC CCL246) were transduced with Av1LacZ4 or Av9LacZ4 at a multiplicity of infection of 5, 10, 100, 500, or 1,000 according to the procedure of Example 1. Transduction data are given in Table III below.

TABLE III

| Av9LacZ4 | | Av1LacZ4 | |
|---|---|---|---|
| (MOI) | | (MOI) | |
| 5 | ++ | 5 | −/+ |
| 10 | +++ | 10 | −/+ |
| 100 | N/A | 100 | −/+ |
| 500 | +++ | 500 | N/A |
| 1,000 | N/A | 1,000 | −/+ |

−/+ 0-25% transduction
++ 25-50% transduction
+++ 50-75% transduction
++++ 75-100% transduction

The results of the experiments in this example suggest that an adenoviral vector having a head portion of the fiber of Adenovirus 3 may be employed in the treatment of leukemias or lymphomas.

### Example 4

### Transduction of Human Smooth Muscle Cells

HISM human intestinal jejunum smooth muscle cells (ATCC CRL-1692) were transduced with Av1LacZ4 or Av9LacZ4 at 10, 100, or 1,000 particles/cell according to the procedure of Example 1. Each experiment was carried out in triplicate, and the percentages of transduced cells (mean +/−standard deviation) are given in Table IV below.

TABLE IV

| Particles/cell | Av9LacZ4 | Av1LacZ4 |
|---|---|---|
| 10 | 13.5 +/− 1.8 | 0.1 +/− 0.1 |
| 100 | 74.3 +/− 2.7 | 0.5 +/− 0.5 |
| 1,000 | 99.0 +/− 3.8 | 7.0 +/− 0.8 |

The above results suggest that an adenovirus having a head portion of the fiber of Adenovirus 3 may be employed in the transduction of smooth muscle cells, such as smooth muscle cells of the digestive system or of the vasculature, and thus such adenoviruses may be useful in the treatment of a variety of disorders, such as the treatment of restenosis or of vascular lesions.

### Example 5

### Transduction of Human Aortic Smooth Muscle Cells

Human aortic smooth muscle cells (Clonetics) were transduced with Av1LacZ4 or Av9LacZ4 at 10, 100, or 1,000 particles/cell according to the procedure of Example 1. Each experiment was carried out in triplicate, and the percentages of transduced cells (mean +/−standard deviation) are given in Table V below.

TABLE V

| Particles/cell | Av9LacZ4 | Av1LacZ4 |
|---|---|---|
| 10 | 2.5 +/− 1.1 | 0 +/− 0 |
| 100 | 11.2 +/− 3.3 | 0.63 +/− 0 |
| 1,000 | 43.8 +/− 5.8 | 0.34 +/− 0.1 |

The above data suggest that an adenoviral vector having the head portion of the fiber of Adenovirus 3 may be employed in the treatment of restenosis following angioplasty for the transduction of vascular smooth muscle cells for the delivery of a therapeutic transgene for the inhibition of smooth muscle cell proliferation.

The disclosures of all patents, publications (including published patent applications), database accession numbers, and depository accession numbers referenced in this specification are specifically incorporated herein by reference in their entirety to the same extent as if each such individual patent, publication, database accession number, and depository accession number were specifically and individually indicated to be incorporated by reference.

It is to be understood, however, that the scope of the present invention is not to be limited to the specific embodiments described above. The invention may be practiced other than as particularly described and still be within the scope of the accompanying claims.

What is claimed is:

1. A method of transferring at least one DNA sequence into cells, comprising:
transducing said cells with a modified adenovirus including said at least one DNA sequence, wherein said adenovirus, prior to modification, is of a first serotype, and wherein, in the modified adenovirus, at least a portion of the fiber of said adenovirus of said first serotype is removed and is replaced with at least a portion of the fiber of an adenovirus of a second serotype, and wherein said cells include a receptor which binds to said at least a portion of the fiber of said adenovirus of said second serotype, and whereby transfer of said at least one DNA sequence into said cells is effected through binding of said modified adenovirus to said cells.

2. The method of claim 1 wherein said fiber of said adenovirus includes a head portion, a shaft portion, and a tail portion, and at least a portion of the head portion of the fiber of said adenovirus of said first serotype is removed and replaced with at least a portion of the head portion of the fiber of said adenovirus of said second serotype.

3. The method of claim 1 wherein said adenovirus of said first serotype is an adenovirus of a serotype within Subgenus C, and said adenovirus of said second serotype is an adenovirus of a serotype within a subgenus selected from the group consisting of Subgenera A, B, D, E, and F.

4. The method of claim 3 wherein said adenovirus of said second serotype is an adenovirus of a serotype within Subgenus B.

5. The method of claim 4 wherein said adenovirus of said first serotype is Adenovirus 5, and said adenovirus of said second serotype is Adenovirus 3.

6. The method of claim 1 wherein said cells are selected from the group consisting of lung cells, hematopoietic cells, lymphoma cells, leukemia cells, smooth muscle cells, and tumor cells.

7. The method of claim 1 wherein said cells are transduced with said modified adenovirus in vivo.

8. The method of claim 6 wherein said cells are lung cells.

9. The method of claim 6 wherein said cells are hematopoietic cells.

10. The method of claim 6 wherein said cells are tumor cells.

11. The method of claim 10 wherein said tumor cells are head and neck cancer cells.

12. The method of claim 10 wherein said tumor cells are neuroblastoma cells.

13. The method of claim 6 wherein said cells are lymphoma cells.

14. The method of claim 6 wherein said cells are leukemia cells.

15. The method of claim 6 wherein said cells are smooth muscle cells.

16. A method of transferring at least one DNA sequence into cells, comprising:
transducing said cells with a modified adenovirus including said at least one DNA sequence, wherein said adenovirus, prior to modification, is of the Adenovirus 5 serotype, and wherein, in the modified adenovirus, the head portion of the fiber of Adenovirus 5 is removed and replaced with the head portion of the fiber of Adenovirus 3, and wherein said cells include a receptor which binds to the head portion of the fiber of Adenovirus 3, and whereby transfer of said at least one DNA sequence into said cells is effected through binding of said modified adenovirus to said cells.

17. A modified adenovirus including at least one DNA sequence to be transferred into cells, wherein said adenovirus, prior to modification, is of the Adenovirus 5 serotype, and wherein, in the modified adenovirus, the head portion of the fiber of Adenovirus 5 is removed and replaced with the head portion of the fiber of Adenovirus 3.

18. A composition, comprising:
the modified adenovirus of claim 17; and a pharmaceutically acceptable carrier.

19. A method of transferring at least one polynucleotide into cells, comprising:
contacting said cells with a gene transfer vehicle other than an adenovirus, said gene transfer vehicle including said at least one polynucleotide, and wherein said gene transfer vehicle includes at least a portion of the fiber of an adenovirus of a desired serotype, and wherein said cells include a receptor which binds to said at least a portion of the fiber of said adenovirus of a desired serotype, and thereby transfer of said at least one polynucleotide into said cells is effected through binding of said gene transfer vehicle to said cells.

20. The method of claim 19 wherein said gene transfer vehicle includes at least a portion of the head portion of the fiber of said adenovirus or a desired serotype.

21. The method of claim 20 wherein said gene transfer vehicle includes the head portion of the fiber of Adenovirus 3.

22. A gene transfer vehicle other than an adenovirus which includes at least a portion of the fiber of an adenovirus of a desired serotype.

23. The gene tranfer vehicle of claim 22 wherein said gene transfer vehicle includes the head portion of the fiber of Adenovirus 3.

24. An adenovirus of the Adenovirus 3 serotype including at least one heterologous DNA sequence.

25. A composition, comprising:
the adenovirus of claim 24; and a pharmaceutically acceptable carrier.

26. A method of transferring at least one heterologous DNA sequence into cells, comprising:
transducing said cells with the adenovirus of claim 24, and wherein said cells include a receptor which binds to the fiber of Adenovirus 3, and whereby transfer of said at least one heterologous DNA sequence is effected through binding of said adenovirus to said cells.

27. A method of transferring at least one polynucleotide into cells, comprising:
contacting said cells with a gene transfer vehicle including said at least one polynucleotide, wherein said gene transfer vehicle includes at least a portion of the fiber of Adenovirus 3, and wherein said cells include a receptor which binds to at least a portion of the fiber of Adenovirus 3, and whereby transfer of said at least one polynucleotide into cells is effected through binding of said gene transfer vehicle to said cells.
